(19) United States
(12) Patent Application Publication
(10) Pub. No.: US 2005/0084929 A1
(43) Pub. Date: Apr. 21, 2005

(54) Human prostaglandin EP4 receptor variants and methods of using same

(76) Inventors: Yanbin Liang, Irvine, CA (US); David F. Woodward, Lake Forest, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 10/688,790

(22) Filed: Oct. 17, 2003

(51) Int. Cl. C07H 21/04; C07K 14/71

(52) U.S. Cl. 435/69.1; 435/320.1; 435/325; 530/350; 536/23.5

(57) ABSTRACT

The present invention provides alternatively spliced EP₄ receptor variants, as well as related nucleic acid molecules and screening methods.

### BACKGROUND OF THE INVENTION

This invention relates generally to molecular medicine and, more specifically, to alternatively spliced prostaglandin EP₄ receptors.

Prostaglandins (PG) and thromboxane, collectively named prostanoids, are oxygenated fatty acids that bind to seven transmembrane domain G-protein coupled receptors (GPCRs). The classification of prostanoid receptors into DP, EP, FP, IP, and TP is based on the binding and functional potency of the five naturally occurring prostanoids, PGD₂, PGE₂, PGF_{2α}, I₂, and TXA₂, respectively. Prostanoid receptors have been cloned and expressed in cultured cells, where ligand binding and signal transduction properties have been studied. It is recognized that prostanoids can bind to more than one prostanoid receptor type; however, each prostanoid binds to its respective receptor with an affinity at least one order of magnitude higher than its affinity for the other four prostanoid receptors.

Prostanoids produce numerous physiologic and pathophysiologic effects and regulate cellular processes in nearly every tissue. The wide spectrum of prostanoid action includes effects on immune, endocrine, cardiovascular, renal and reproductive systems as well as the contraction and relaxation of smooth muscle. Accordingly, prostanoids and prostanoid analogues have been used as drugs to treat a variety of clinical conditions, including, but not limited to, various types of pain.

Although the broad classification of prostanoid receptors into five classes remains intact, the differential effects of PGE₂ on target tissues provides evidence for a subdivision within the EP receptor family. To date, four subtypes of EP receptors, termed EP₁, EP₂, EP₃, and EP₄ have been cloned and expressed. EP₄ receptors are expressed in a range of different species and in a several tissues, including, without limitation, liver, adrenal gland, thymus, ileum, spleen, adipose tissue, heart, uterus, testis, prostate, kidney, lung and colon, and in B cells and carcinoma cells. Recently, roles for the EP₄ receptor in vasodilatation of venous and arterial beds, colon cancer and osteoporosis have been reported.

Although some EP receptor subtype selective ligands exist, many of these compounds act at multiple PG receptor subtypes. Thus, while compounds have been synthesized that have reduced agonist activity at other prostanoid receptors, currently available drugs still have some agonist activity at other receptors. This lack of receptor specificity can result in undesirable side effects.

A goal of clinical pharmacology and the pharmaceutical industry is the development of more selective drugs with greater efficacy and fewer side effects than those currently in use. In order to more effectively treat conditions such as high blood pressure, colon cancer or osteoporosis where EP₄ receptor modulators can be of benefit, new receptors related to the known wild-type EP₄ receptor must be discovered and used to design screening assays for identification of compounds that bind more specifically to the known EP₄ receptor. Newly identified EP₄ receptors such as alternatively spliced EP₄ receptors also can be more closely associated with a disease such as such as high blood pressure, colon cancer or osteoporosis than the known EP₄ receptor and can be targets for drug discovery efforts, resulting in the development of drugs having greater efficacy or fewer side effects than drugs developed against the known wild-type EP₄ receptor.

Thus, there exists a need for the discovery of new EP₄ receptors which can be used, for example, to design more specific drugs with fewer side effects. The present invention satisfies this need and provides related advantages as well.

### SUMMARY OF THE INVENTION

The present invention provides an isolated polypeptide that contains the amino acid sequence of SEQ ID NO: 10 or SEQ ID NO: 16. Cells which contain such an exogenously expressed polypeptide also are provided.

The present invention further provides an isolated polypeptide containing an amino acid sequence having at least 50% amino acid identity with SEQ ID NO: 18, and containing the amino acid sequence of SEQ ID NO: 8, 10, 12, 14 or 16, or a conservative variant of SEQ ID NO: 8, 10, 12, 14 or 16. Cells containing such an exogenously expressed polypeptide also are provided.

Further provided herein is an isolated polypeptide that contains the amino acid sequence of SEQ ID NO: 2, 4 or 6, or a conservative variant thereof. Such a polypeptide can contain, for example, the amino acid sequence of SEQ ID NO: 2, 4 or 6, or can consist of the amino acid sequence of SEQ ID NO: 2, 4 or 6. The invention also provides cells containing such an exogenously expressed polypeptide.

Also provided herein is an EP₄ receptor variant binding agent which binds SEQ ID NO: 10, or an epitope thereof. Such a binding agent can be, for example, an an antibody, or antigen binding fragment thereof.

The present invention additionally provides a method for identifying a compound that modulates an EP₄ receptor variant by contacting an isolated EP₄ receptor variant or an EP₄ receptor variant over-expressed in a genetically engineered cell with a compound, and determining the level of an indicator that correlates with modulation of the EP₄ receptor variant, where an alteration in the level of the indicator as compared to a control level indicates that the compound is a compound that modulates the EP₄ receptor variant. Such an alteration can be, for example, an increase or decrease in the level of the indicator. Useful indicators include, but are not limited to, calcium. Any of the above polypeptides can be useful in the methods of the invention for identifying a compound that modulates an EP₄ receptor variant. Furthermore, compounds to be screened according to a method of the invention include, without limitation, polypeptides and small molecules.

The present invention further provides a method for identifying a compound that specifically binds to an EP₄ receptor variant by contacting an isolated EP₄ receptor variant or an EP₄ receptor variant over-expressed in a genetically engineered cell with a compound, and determining specific binding of the compound to the EP₄ receptor variant. Any of the above polypeptides, in isolated form or over-expressed in a genetically exgineered cell, can be useful in the screening methods of the invention. Compounds to be screened according to a method of the invention include, without limitation, polypeptides and small molecules. In one embodiment, the receptor variant is contacted with a compound in vitro.

The present invention further provides a method for identifying a compound that differentially modulates an EP₄ receptor variant by contacting an isolated EP₄ receptor variant or an EP₄ receptor variant over-expressed in a genetically engineered cell with a compound; determining the level of an indicator which correlates with modulation of the EP₄ receptor variant; contacting a second receptor with the compound; determining the level of a corresponding indicator which correlates with modulation of the second receptor; and comparing the level of the indicator and corresponding indicator, where a different level of the indicator compared to the level of the corresponding indicator indicates that the compound is a compound that differentially modulates the EP₄ receptor variant. The second receptor can be, for example, a different EP₄ receptor variant such as any of the polypeptides disclosed hereinabove, or can be, for example, a receptor having the amino acid sequence of SEQ ID NO: 18, or a functional fragment thereof. Any of a variety of indicators are useful in the screening methods of the invention including, but not limited to, calcium. Furthermore, compounds to be screened according to a method of the invention include, without limitation, polypeptides and small molecules.

Further provided herein is a method for identifying a compound that differentially binds to an EP₄ receptor variant by contacting an isolated EP₄ receptor or an EP₄ receptor variant over-expressed in a genetically engineered cell with a compound; determining specific binding of the compound to the EP₄ receptor variant; contacting a second receptor with the compound; determining specific binding of the compound to the second receptor; and comparing the level of specific binding to the EP₄ receptor variant with the level of specific binding to the second receptor, where a different level of specific binding to the EP₄ receptor variant as compared to the level of specific binding to the second receptor indicates that the compound is a compound that differentially binds to the EP₄ receptor variant. In such a screening method of the invention, the second receptor can be, for example, a different EP₄ receptor variant such as any of the polypeptides disclosed hereinabove. The second receptor also can be, for example, a polypeptide containing the amino acid sequence of SEQ ID NO: 18, or a functional fragment thereof. In a screening method of the invention, the contacting can occur, for example, in vitro, and compounds to be screened include, without limitation, polypeptides and small molecules.

The present invention further provides an isolated nucleic acid molecule which includes a nucleotide sequence that encodes a polypeptide containing an amino acid sequence having at least 50% amino acid identity with SEQ ID NO: 18, and containing the amino acid sequence of SEQ ID NO: 8, 10, 12, 14 or 16, or a conservative variant thereof. Such a nucleic acid molecule can include, for example, a nucleotide sequence that encodes the amino acid sequence of SEQ ID NO: 2, 4 or 6, or a conservative variant thereof. In one embodiment, such an isolated nucleic acid molecule includes a nucleotide sequence that encodes the amino acid sequence of SEQ ID NO: 2, 4 or 6. In another embodiment, such an isolated nucleic acid molecule consists of a nucleotide sequence that encodes the amino acid sequence of SEQ ID NO: 2, 4 or 6. In still a further embodiment, an isolated nucleic acid molecule of the invention contains the nucleotide sequence of SEQ ID NO: 1, 3 or 5. Vectors containing any of the nucleic acid molecules of the invention, as well as host cells containing such vectors, further are provided herein.

### BRIEF DESCRIPTION OF THE DRAWINGS

FIG. 1 shows the nucleotide sequence of EP₄ receptor variant VAR-1 (SEQ ID NO: 1). The underlined sequence indicates novel nucleotide sequence compared to the nucleotide sequence of the known wild-type human EP₄ receptor (SEQ ID NO: 17). The start and stop codons for EP₄ receptor variant VAR-1 are indicated in bolded capital letters.

FIG. 2 shows the nucleotide sequence of EP₄ receptor variant VAR-2 (SEQ ID NO: 3). The underlined sequence indicates novel nucleotide sequence compared to the nucleotide sequence of the known wild-type human EP₄ receptor (SEQ ID NO: 17). The start and stop codons for EP₄ receptor variant VAR-2 are indicated in bolded capital letters.

FIG. 3 shows the nucleotide sequence of EP₄ receptor variant VAR-3 (SEQ ID NO: 5). The underlined sequence indicates novel nucleotide sequence compared to the nucleotide sequence of the known wild-type human EP₄ receptor (SEQ ID NO: 17). The start and stop codons for EP₄ receptor variant VAR-3 are indicated in bolded capital letters.

FIG. 4 shows a comparison of the amino acid sequences of the known wild-type human EP₄ receptor (SEQ ID NO: 18), abbreviated as EP4 WT, with human EP₄ receptor variants VAR-1 (SEQ ID NO: 2), VAR-2 (SEQ ID NO: 4), and VAR-3 (SEQ ID NO: 6). The bolded sequence at the carboxy termini of VAR-1, VAR-2 and VAR-3 indicate differences between these sequences and the wild-type human EP₄ receptor sequence.

FIG. 5 shows the intron/exon structure of human EP₄ receptor genomic DNA clone AC 093264.2 and the EP₄ receptor variants VAR-1, VAR-2, and VAR-3. Exons 2 and 3 of the human wild-type EP₄ receptor genomic DNA are shown as well as the location of alternatively spliced exon A, which is present in VAR-1, alternatively spliced exon C, which is present in VAR-2, and alternatively spliced exons B and C, which are present in VAR-3. Intronic sequence is shown as a thin line, while exonic sequence is shown as a thick line.

FIG. 6 shows distribution of mRNA from EP₄ receptor variants VAR-1, VAR-2, and VAR-3 mRNA in various tissues using multiple tissue RT-PCR analysis. The location of PCR products of the correct size for EP₄ receptor variants VAR-1 through VAR-3 are indicated by arrows.

### DETAILED DESCRIPTION OF THE INVENTION

The present invention is directed to the exciting discovery of several novel EP₄ receptor variants. Such EP₄ receptor variants can be used to determine and refine the specificity of binding of compounds that bind to the known wild-type EP₄ receptor. These EP₄ receptor variants also can be used to identify compounds that differentially modulate or bind to a first EP₄ receptor variant in relation to a second EP₄ receptor variant or wild-type EP₄ receptor. Such a compound can be, for example, a ligand that specifically binds to a novel EP₄ receptor variant disclosed herein.

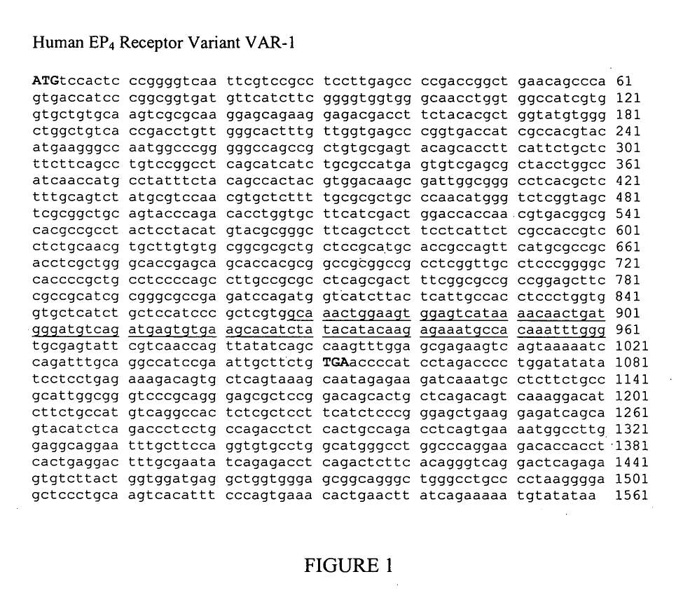

As disclosed herein in Example I, several novel EP₄ receptor variants were identified using the reverse transcription polymerase chain reaction (RT-PCR) and the following EP₄ receptor primers: AGATGGTCATCTTACTCATTG (SEQ ID NO: 19) and GATGTACTGCTGATCTCCTTC (SEQ ID NO: 20). In particular, three novel alternatively spliced EP₄ receptor variants, referred to herein as human EP₄ receptor variants VAR-1 through VAR-3, were identified as distinct from the wild-type human EP₄ receptor (see FIGS. 1-4).

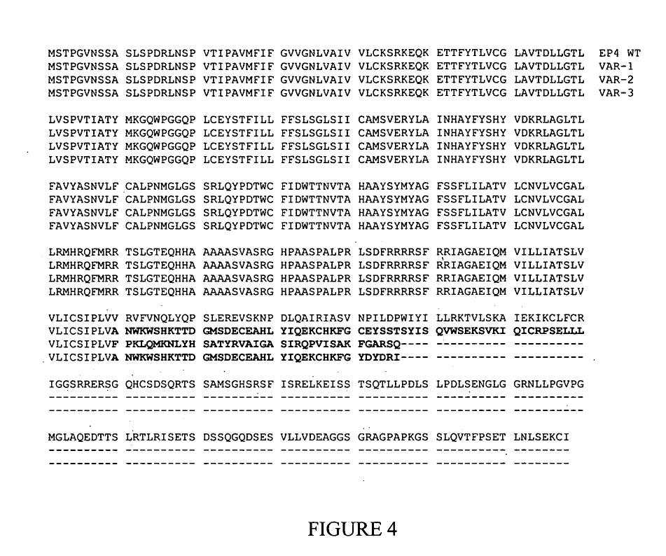

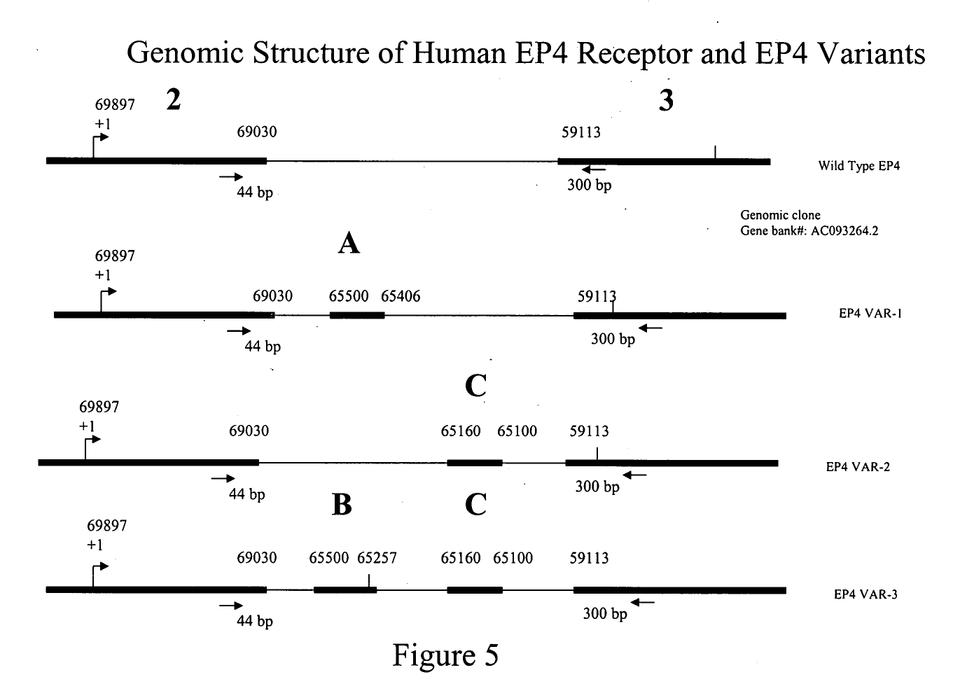

As further disclosed herein, sequence analysis of nucleic acid molecules encoding the alternatively spliced EP₄ receptor variants revealed novel carboxy-terminal amino acid sequences. The amino acid sequences of the wild-type human EP₄ receptor and the alternatively spliced EP₄ receptor variants are shown in FIG. 4. As shown in FIG. 5, exons 2 and 3 are conserved between the known wild-type EP₄ receptor and the alternatively spliced EP₄ receptors while the alternatively spliced EP₄ receptors additionally contain one or more alternatively spliced exons (exons A, B, or C) not present in the wild type receptor.

Comparison of the known wild-type human EP₄ receptor amino acid sequence (SEQ ID NO: 18) to the alternatively spliced human EP₄ receptor variants revealed the amino acid sequence at the junction between conserved exon 2 and the relevant alternatively spliced exon (exon A within EP₄ receptor variant VAR-1 or exon B within VAR-3) to be IPLVANWKW (SEQ ID NO: 14), where the first four amino acids correspond to amino acid sequence present in conserved exon 2 and the remaining five amino acids are residues derived from newly identified exon A which is present in EP₄ receptor variant VAR-1 (see FIGS. 4 and 5). Similarly, the amino acid sequence at the junction between conserved exon 2 and the relevant alternatively spliced exon (exon C) within EP₄ receptor variant VAR-2 is IPLVFPKLQ (SEQ ID NO: 16), where the first four amino acids correspond to the amino acid sequence present in conserved exon 2 and the remaining five amino acids are residues derived from the newly identified exon C.

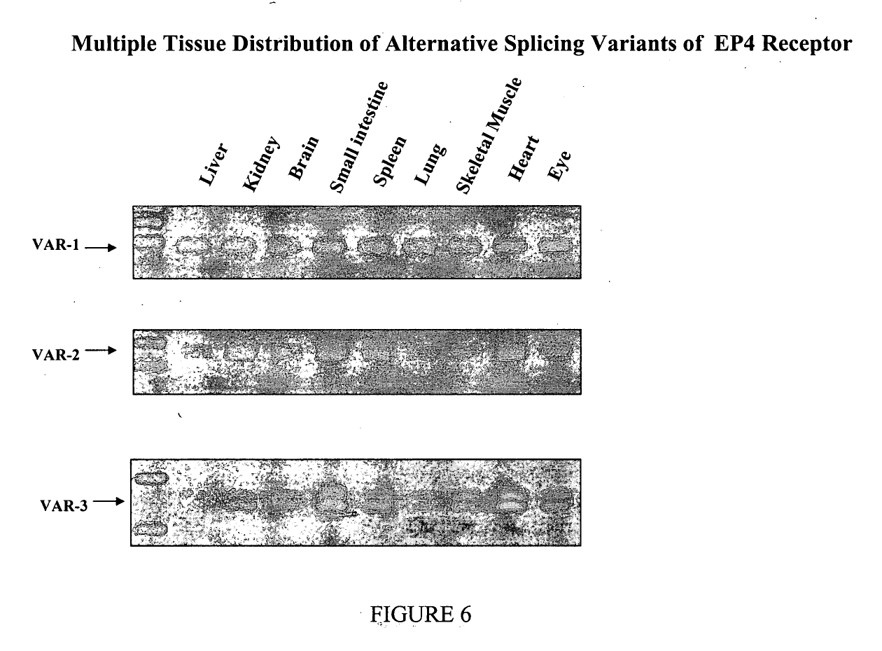

As further disclosed herein in FIG. 4, the carboxy terminal amino acid sequence in EP₄ receptor variants VAR-1 through VAR-3 is unique to the respective EP₄ receptor variant. Specifically, the unique carboxy terminus of EP₄ receptor variant VAR-1 has the amino acid sequence ANWKWSHKTTDGMSDECEAHLYIQEKCHKFGCEYSSTSYISQVW SEKSVKIQICRPSELLL (SEQ ID NO: 8); the unique carboxy terminus of EP₄ receptor variant VAR-2 has the amino acid sequence FPKLQMKNLYHSATYRVAIGASIRQPVISAKFGARSQ (SEQ ID NO: 10); and the unique carboxy terminus of EP₄ receptor Variant VAR-3 has the amino acid sequence ANWKWSHKTTDGMSDECEAHLYIQEKCHKFGYDYDRI (SEQ ID NO: 12). Furthermore, expression of alternatively spliced human EP₄ receptor variants VAR-1 through VAR-3 can be found in a variety of tissues including liver, kidney, brain, small intestine, spleen, lung, skeletal muscle, heart and eye (see FIG. 6 and Example II).

Based on these discoveries, the present invention provides novel, alternatively spliced EP₄ receptor variants and screening methods that rely on these variants. In particular, the invention provides an isolated polypeptide containing SEQ ID NO: 10 or 16, which represent the unique carboxy-terminal and junctional portions of newly identified EP₄ variant VAR-2. The present invention further provides an isolated polypeptide containing an amino acid sequence having at least 50% amino acid identity with SEQ ID NO: 18 and containing the amino acid sequence of SEQ ID NO: 8, 10, 12, 14, or 16, or a conservative variant thereof. Also provided herein is an isolated polypeptide containing the amino acid sequence of SEQ ID NO: 2, 4 or 6, or a conservative variant thereof. The invention further provides an isolated polypeptide containing or consisting of the amino acid sequence of SEQ ID NO: 2, 4 or 6.

The present invention further provides a method for identifying a compound that modulates an EP₄ receptor variant by contacting an isolated EP₄ receptor variant or an EP₄ receptor variant over-expressed in a genetically engineered cell with a compound and determining the level of an indicator which correlates with modulation of the EP₄ receptor variant, where an alteration in the level of the indicator as compared to a control level indicates that the compound is a compound that modulates the EP₄ receptor variant. The present invention also provides a method for identifying a compound that specifically binds to an EP₄ receptor variant by contacting an isolated EP₄ receptor variant or an EP₄ receptor variant over-expressed in a genetically engineered cell with a compound and determining specific binding of the compound to the EP₄ receptor variant.

The invention further provides a method for identifying a compound that differentially modulates an EP₄ receptor variant by a) contacting an isolated EP₄ receptor variant or an EP₄ receptor variant over-expressed in a genetically engineered cell with a compound; b) determining the level of an indicator which correlates with modulation of the EP₄ receptor variant; c) contacting a second receptor with the compound; d) determining the level of a corresponding indicator after contacting of the compound to the second receptor; and e) comparing the level of the indicator from step (b) with the level of the corresponding indicator from step (d), where a different level of the indicator from step (b) compared to the level of the corresponding indicator from step (d) indicates that the compound is a compound that differentially modulates the EP₄ receptor variant.

Further provided herein is a method for identifying a compound that differentially binds to an EP₄ receptor variant by a) contacting an isolated EP₄ receptor variant or an EP₄ receptor variant over-expressed in a genetically engineered cell with a compound; b) determining specific binding of the compound to the EP₄ receptor variant; c) contacting a second receptor with the compound; d) determining specific binding of the compound to the second receptor; and e) comparing the level of specific binding from step (b) with the level of specific binding from step (d), where a different level of specific binding from step (b) compared to the level of specific binding from step (d) indicates that the compound is a compound that differentially binds to the EP₄ receptor variant.

The methods of the invention can be useful for designing drugs that bind to or modulate the wild-type human EP₄ receptor (SEQ ID NO: 18) in preference to one or more alternatively spliced variants or for identifying compounds that bind to or modulate one or more EP₄ receptor variants in preference to other EP₄ receptor variants or the wild type EP₄ receptor. Compounds identified by a method of the invention can be therapeutically useful in preventing or reducing the severity of a condition where modulation of an EP₄ receptor or an EP₄ receptor variant is beneficial.

The EP₄ receptor is one of four prostaglandin E receptor subtypes activated by prostaglandin E2 The human EP₄ receptor cDNA encodes a 488 amino acid polypeptide with a predicted molecular mass of about 53 kD. Prior to 1995, the EP₄ receptor was generally referred to as the EP₂ receptor. See Foord et al., Genomics 35:182-188 (1996), for a more complete discussion of EP₄ nomenclature.

The EP₄ receptor is expressed in a variety of tissues including lung, peripheral blood lymphocytes, and vasculature. Foord et al., supra, 1996, isolated the EP₄ receptor gene and showed that it consists of 3 exons and spans about 22 kb of genomic DNA. The first exon is non-coding. Exons 2 and 3 encode a predicted 488 amino acid protein. The gene structure resembles that of the thromboxane receptor, PGI receptor, and PGD receptor. The promoter region contains motifs found in other cytokine-activated genes. Foord et al., supra, 1996, also observed two apparent pseudogenes of the EP₄ receptor. Duncan et al. mapped the PTGER4 gene, which they referred to as PTGER2, to 5p13.1 by in situ hybridization (Duncan et al., Genomics 25:740-742 (1995)).

Mori et al. examined the expression of the human EP₄ receptor in cells of the immune system (Mori et al., J. Molec. Med. 74:333-336 (1996)). The level of EP₄ receptor in T-cell lines was downregulated by phorbol 12-myristate 13-acetate (PMA), an activator of protein kinase C. However, in Raji and monocytoid cell lines, marked upregulation of EP₄ receptor was observed in response to PMA, while levels of other PGE receptors remained unchanged.

Recent work indicates that prostaglandin E2 synthesis, as well as positive regulatory cyclooxygenase (COX) enzyme products, are upregulated in cervical cancer. For example, real-time quantitative PCR and Western blot analysis demonstrated COX2 RNA and protein expression in squamous cell carcinomas and adenocarcinomas while minimal expression of COX2 was detected in histologically normal cervix (Sales et al., J. Clin. Endocr. Metab. 86:2243-2249 (2001)). Immunohistochemical analyses also localized prostaglandin E2 (PGE₂) and COX2 expression synthesis to neoplastic epithelial cells in a variety of squamous cell carcinomas and adenocarcinomas. Immunoreactive COX2 and PGE₂ were also colocalized to endothelial cells lining the microvasculature.

To establish whether PGE₂ has an autocrine/paracrine effect in cervical carcinomas, the expression of two PGE₂ receptor subtypes, namely EP₂ and EP₄, was analyzed by real-time quantitative PCR. Expression of EP₂ and EP₄ receptors was significantly higher in carcinoma tissue than in histologically normal cervix, indicating that COX2, EP₂, and EP₄ expression and PGE₂ synthesis are upregulated in cervical cancer tissue and that PGE₂ can regulate neoplastic cell function in cervical carcinomas, for example, via the EP₄ receptor. In addition, PGE₂ has been shown to contribute to colon carcinogenesis through EP₁ and EP₄ receptors, indicating that antagonists of these receptors act as chemopreventative agents against colon cancer (Mutoh et al., Cancer Res. 62:28-32 (2002), Fujino et al., J. Biol. Chem. 277:2614-2619 (2002), and Sheng et al., J. Biol. Chem. 276:18075-19081 (2001)).

Mice deficient in the prostaglandin receptor EP₄ have been generated using targeted disruption (Segi et al., Biochem. Biophys. Res. Comm. 246: 7-12 (1998)). Loss of the EP₄ receptor was not lethal in utero, but most EP₄ −/− neonates became pale and lethargic approximately 24 hours after birth and died within 72 hours. Fewer than 5% of the EP₄ −/− mice survived and grew normally for more than 1 year. Histologic examination revealed that the ductus arteriosus in dead neonates remained open, while it was partially closed in the survivors. Furthermore, in situ hybridization studies showed that EP₄ mRNA was strongly expressed in the ductus in wild-type mice. These results indicate that neonatal death was at least partly due to patent ductus arteriosus and that the EP₄ receptor plays a role in regulation of the patency of this vessel. These results further indicate that normal function of the EP₄ receptor is essential in neonatal adaptation of the circulatory system.

Prostaglandin E2 is known to promote both bone resorption and bone formation. Furthermore, analysis of mice deficient in each of the four EP receptor subtypes showed that the EP₄ receptor mediates the bone-forming activity of PGE₂ (Yoshida et al., Proc. Nat. Acad. Sci. 99: 4580-4585 (2002)). Consistently, bone formation was induced in wild-type mice by infusion of EP₄-selective agonists and not by agonists specific for other EP subtypes. In cultures of bone marrow cells from wild-type mice, PGE₂ induced expression of core-binding factor alpha-1 and enhanced formation of mineralized nodules, both of which were absent in cell cultures from EP₄-deficient mice. Furthermore, administration of EP₄ agonists restored bone mass and strength normally lost in rats subjected to ovariectomy or immobilization. Histomorphometric analysis revealed that EP₄ agonists induced significant increases in the volume of cancellous bone, osteoid formation, and the number of osteoblasts in the affected bone of immobilized rats, indicating that activation of EP₄ induces de novo bone formation. In addition, osteoclasts were found on the increased bone surface at a density comparable to that found in the bone of control animals. These results indicate that activation of the EP₄ receptor induces bone remodeling in vivo and that EP₄-selective drugs may be beneficial in humans with osteoporosis.

The EP₄ receptor also plays a role in immune regulation. For example, the migration of Langerhans cells, which express all four EP receptor subtypes, to regional lymph nodes was decreased only in EP₄-deficient (Ptger4−/−) mice and in wild-type mice treated with an EP₄ antagonist (Kabashima et al., Nature Med. 9: 744-749 (2003)). In addition, an EP₄ agonist promoted the migration of Langerhans cells, increased the expression of co-stimulatory molecules on these cells, and enhanced the stimulation of T cells by Langerhans cells in mixed lymphocyte reaction in vitro. Contact hypersensitivity to antigen also was impaired in Ptger4−/− mice and in wild-type mice treated with an EP₄ antagonist during sensitization. Thus, PGE₂-EP₄ signaling can facilitate initiation of skin immune responses by promoting the migration and maturation of Langerhans cells. The present invention relates to novel EP₄ receptor variants related to the wild-type EP₄ receptor, which has been cloned from several species including mouse (GenBank Accession No. P32240), rat (GenBank Accession No. NP_{—}114465.1), and human (GenBank Accession No. NP_{—}000949.1). In particular, the invention provides novel EP₄ receptor variants which are alternatively spliced forms of the human wild-type EP₄ receptor. In one embodiment, the invention provides an isolated polypeptide containing SEQ ID NO: 10 or 16. In another embodiment, the invention provides an isolated polypeptide containing an amino acid sequence having at least 50% amino acid identity with SEQ ID NO: 18 and containing the amino acid sequence of SEQ ID NO: 8, 10, 12, 14 or 16, or a conservative variant of SEQ ID NO: 8, 10, 12, 14 or 16. In further embodiments, the invention provides an isolated polypeptide containing the amino acid sequence of SEQ ID NO: 2, 4 or 6, or a conservative variant of SEQ ID NO: 2, 4 or 6. In yet further embodiments, the invention provides an isolated polypeptide that contains or consists of the amino acid sequence of SEQ ID NO: 2, 4 or 6.

The invention further provides an EP₄ receptor variant binding agent which binds the amino acid sequence of SEQ ID NO: 10, or an epitope thereof. Such an EP₄ receptor variant binding agent can be, without limitation, an antibody or antigen binding fragment thereof. The invention additionally provides a cell that includes an exogenously expressed polypeptide containing the amino acid sequence of SEQ ID NO: 10 or 16; a cell which includes an exogenously expressed polypeptide containing an amino acid sequence having at least 50% amino acid identity with SEQ ID NO: 18 and containing the amino acid sequence of SEQ ID NO: 8, 10, 12, 14 or 16, or a conservative variant thereof; and a cell which includes an exogenously expressed polypeptide containing the amino acid sequence of SEQ ID NO: 2, 4 or 6, or a conservative variant thereof.

The present invention also provides a method for identifying a compound that modulates an EP₄ receptor variant by contacting an isolated EP₄ receptor variant or an EP₄ receptor variant over-expressed in a genetically engineered cell with a compound and determining the level of an indicator which correlates with modulation of the EP₄ receptor variant, where an alteration in the level of the indicator as compared to a control level indicates that the compound is a compound that modulates the EP₄ receptor variant. The alteration can be, for example, an increase or decrease in the level of an indicator such as, without limitation, calcium. A method of the invention can be practiced with any of a variety of EP₄ receptor variants such as an isolated polypeptide containing the amino acid sequence of SEQ ID NO: 10 or 16; an isolated polypeptide containing an amino acid sequence having at least 50% amino acid identity with SEQ ID NO: 18 and containing the amino acid sequence of SEQ ID NO: 8, 10, 12, 14 or 16, or a conservative variant thereof; or an isolated polypeptide containing the amino acid sequence of SEQ ID NO: 2, 4 or 6, or a conservative variant thereof. A method of the invention also can be practiced using any of a variety of EP₄ receptor variants over-expressed in a genetically engineered cell. In one embodiment, the EP₄ receptor variant is exogenously over-expressed in the genetically engineered cell. A variety of compounds can be screened according to the methods of the invention including, but not limited to, polypeptides and small molecules.

The present invention further provides a method for identifying a compound that specifically binds to an EP₄ receptor variant by contacting an isolated EP₄ receptor variant or an EP₄ receptor variant over-expressed in a genetically engineered cell with a compound and determining specific binding of the compound to the EP₄ receptor variant. In particular embodiments, a method of the invention can be practiced with an isolated polypeptide containing the amino acid sequence of SEQ ID NO: 10 or 16; an isolated polypeptide containing an amino acid sequence having at least 50% amino acid identity with SEQ ID NO: 18 and containing the amino acid sequence of SEQ ID NO: 8, 10, 12, 14 or 16, or a conservative variant thereof; or an isolated polypeptide containing the amino acid sequence of SEQ ID NO: 2, 4 or 6, or a conservative variant thereof. In another embodiment, a method of the invention is practiced using an EP₄ receptor variant over-expressed in a genetically engineered cell, for example, an EP₄ receptor variant exogenously over-expressed in a genetically engineered cell. In the methods of the invention, contacting can occur in vivo or in vitro, and the compounds to be screened can include, without limitation, polypeptides and small molecules.

The invention further provides a method for identifying a compound that differentially modulates an EP₄ receptor variant by a) contacting an isolated EP₄ receptor variant or an EP₄ receptor variant over-expressed in a genetically engineered cell with a compound; b) determining the level of an indicator which correlates with modulation of the EP₄ receptor variant; c) contacting a second receptor with the compound; d) determining the level of a corresponding indicator after contacting of the compound to the second receptor; and e) comparing the level of the indicator from step (b) with the level of the corresponding indicator from step (d), where a different level of the indicator from step (b) compared to the level of the corresponding indicator from step (d) indicates that the compound is a compound that differentially modulates the EP₄ receptor variant. The second receptor can be, for example, a distinct EP₄ receptor variant or a wild-type EP₄ receptor from the same or a different species, or a functional fragment thereof. The level of the indicator from step (b) can be greater or less than the level of the indicator from step (d) and the indicator can be, without limitation, calcium. In particular embodiments, a method of the invention is practiced with an isolated polypeptide containing the amino acid sequence of SEQ ID NO: 10 or 16; an isolated polypeptide containing an amino acid sequence having at least 50% amino acid identity with SEQ ID NO: 18 and containing the amino acid sequence of SEQ ID NO: 8, 10, 12, 14 or 16, or a conservative variant thereof; or an isolated polypeptide containing the amino acid sequence of SEQ ID NO: 2, 4 or 6, or a conservative variant thereof. In another embodiment, a method of the invention is practiced using an EP₄ receptor variant over-expressed in a genetically engineered cell, for example, an EP₄ receptor variant exogenously over-expressed in a genetically engineered cell. In the methods of the invention, the compounds to be screened can include, without limitation, polypeptides and small molecules.

The invention further provides a method for identifying a compound that differentially binds to an EP₄ receptor variant by a) contacting an isolated EP₄ receptor variant or an EP₄ receptor variant over-expressed in a genetically engineered cell with a compound; b) determining specific binding of the compound to the EP₄ receptor variant; c) contacting a second receptor with the compound; d) determining specific binding of the compound to the second receptor; and e) comparing the level of specific binding from step (b) with the level of specific binding from step (d), where a different level of specific binding from step (b) compared to the level of specific binding from step (d) indicates that the compound is a compound that differentially binds to the EP₄ receptor variant. The second receptor can be, for example, a distinct EP₄ receptor variant or a wild-type EP₄ receptor from the same or a different species, or a functional fragment thereof. The different level of specific binding can be an increased or decreased level. In particular embodiments, a method of the invention is practiced with an isolated polypeptide containing the amino acid sequence of SEQ ID NO: 10 or 16; an isolated polypeptide containing an amino acid sequence having at least 50% amino acid identity with SEQ ID NO: 18 and containing the amino acid sequence of SEQ ID NO: 8, 10, 12, 14 or 16, or a conservative variant thereof; or an isolated polypeptide containing the amino acid sequence of SEQ ID NO: 2, 4 or 6, or a conservative variant thereof. In another embodiment, a method of the invention is practiced using an EP₄ receptor variant over-expressed in a genetically engineered cell, for example, an EP₄ receptor variant exogenously over-expressed in a genetically engineered cell. In the methods of the invention, contacting can occur in vivo or in vitro, and the compounds to be screened can include, without limitation, polypeptides and small molecules.

The invention further provides an isolated nucleic acid molecule having a nucleotide sequence that encodes a polypeptide containing the amino acid sequence of SEQ ID NO: 10 or 16. The invention also provides an isolated nucleic acid molecule having a nucleotide sequence that encodes a polypeptide containing an amino acid sequence having at least 50% amino acid identity with SEQ ID NO: 18 and containing the amino acid sequence of SEQ ID NO: 8, 10, 12, 14 or 16, or a conservative variant thereof. The invention further provides an isolated nucleic acid molecule containing a nucleotide sequence that encodes the amino acid sequence of SEQ ID NO: 2, 4 or 6, or a conservative variant thereof. For example, the invention provides an isolated nucleic acid molecule containing the nucleotide sequence of SEQ ID NO: 1, 3 or 5. The invention further provides a vector containing a nucleic acid molecule having a nucleotide sequence that encodes a polypeptide containing an amino acid sequence having at least 50% amino acid identity with SEQ ID NO: 18 and containing the amino acid sequence of SEQ ID NO: 8, 10, 12, 14 or 16, or a conservative variant thereof; or a nucleic acid molecule containing a nucleotide sequence that encodes the amino acid sequence of SEQ ID NO: 2, 4 or 6, or a conservative variant thereof. Host cells containing such vectors are further provided herein.

The invention relates, in part, to the identification of novel EP₄ receptor variants. As used herein, the term “EP₄ receptor variant” means a polypeptide containing an amino acid sequence that has at least 30% amino acid identity with the wild-type human EP₄ receptor SEQ ID NO: 18 and further containing the amino acid sequence of SEQ ID NO: 8, 10, 12, 14 or 16, or a conservative variant of SEQ ID NO: 8, 10, 12, 14 or 16. An EP₄ receptor variant can contain an amino acid sequence having, for example, at least 30% amino acid identity, at least 40% amino acid identity at least 50% amino acid identity, at least 60% amino acid identity, at least 70% amino acid identity, at least 80% amino acid identity, at least 90% amino acid identity, or at least 95% amino acid identity with the wild-type human EP₄ receptor SEQ ID NO: 18. As a non-limiting example, an EP₄ receptor variant can contain an amino acid sequence having at least 50% amino acid identity with SEQ ID NO: 18 and containing the amino acid sequence of SEQ ID NO: 8, 10, 12, 14 or 16, or a conservative variant thereof.

Based on the above, it is understood that species homologs of EP₄ receptor variants that contain the amino acid sequence of SEQ ID NO: 8, 10, 12, 14 or 16, or a conservative variant thereof, are encompassed by the definition of EP₄ receptor variant as used herein. As non-limiting examples, an isolated polypeptide containing the amino acid sequence of SEQ ID NO: 2, 4 or 6, or consisting of the amino acid sequence of SEQ ID NO: 2, 4 or 6 is an EP₄ receptor variant of the invention.

An EP₄ receptor variant differs from the known wild-type human EP₄ receptor polypeptide by containing the amino acid sequence of SEQ ID NO: 8, 10, 12, 14 or 16, or a conservative variant of such an amino acid sequence. As used herein in reference to a specified amino acid sequence such as one of SEQ ID NOS: 2, 4, 6, 8, 10, 12, 14 or 16, a “conservative variant” is a sequence in which a first amino acid is replaced by another amino acid or amino acid analog having at least one biochemical property similar to that of the first amino acid; similar properties include, yet are not limited to, similar size, charge, hydrophobicity or hydrogen-bonding capacity.

As an example, a conservative variant can be a sequence in which a first uncharged polar amino acid is conservatively substituted with a second (non-identical) uncharged polar amino acid such as cysteine, serine, threonine, tyrosine, glycine, glutamine or asparagine or an analog thereof. A conservative variant also can be a sequence in which a first basic amino acid is conservatively substituted with a second basic amino acid such as arginine, lysine, histidine, 5-hydroxylysine, N-methyllysine or an analog thereof. Similarly, a conservative variant can be a sequence in which a first hydrophobic amino acid is conservatively substituted with a second hydrophobic amino acid such as alanine, valine, leucine, isoleucine, proline, methionine, phenylalanine or tryptophan or an analog thereof. In the same way, a conservative variant can be a sequence in which a first acidic amino acid is conservatively substituted with a second acidic amino acid such as aspartic acid or glutamic acid or an analog thereof; a sequence in which an aromatic amino acid such as phenylalanine is conservatively substituted with a second aromatic amino acid or amino acid analog, for example, tyrosine; or a sequence in which a first relatively small amino acid such as alanine is substituted with a second relatively small amino acid or amino acid analog such as glycine or valine or an analog thereof. It is understood that a conservative variant of one of SEQ ID NOS: 2, 4, 6, 8, 10, 12, 14 or 16 can have one, two, three, four, five, six, ten, or more amino acid substitutions relative to the specified sequence and that such a conservative variant can include naturally and non-naturally occurring amino acid analogs.

The invention further provides functional fragments of an EP₄ receptor variant containing the amino acid sequence of SEQ ID NO: 8, 10, 12, 14 or 16. As non-limiting examples, a functional fragment of an EP₄ receptor variant such as a ligand-binding fragment or a fragment of an EP₄ receptor variant that is involved in signal transduction can be useful in a method of the invention in place of the full-length EP₄ receptor variant. As non-limiting examples, functional ligand-binding domain fragments can be fragments including the seven transmembrane helices and can further include, for example, the second extracellular loop or the carboxy terminus (C-terminal 156 residues), as described in Stillman et al., Eur. J. Pharm. 357:73-82 (1998), and Bastepe and Ashby, Mol. Pharm. 51:343-349 (1997). As further understood by one skilled in the art, an EP₄ receptor variant can optionally include non-homologous amino acid sequence. As non-limiting examples, an EP₄ receptor variant can contain an epitope tag or can be fused to a non-homologous polypeptide such as gluthionine S-transferase.

As discussed above, the EP₄ receptor variants VAR-1 through VAR-3 contain amino acid sequence that is not present in the wild-type EP₄ receptor SEQ ID NO: 18 (see FIGS. 4 and 5). For example, the alternatively spliced EP₄ receptor variants VAR-1, VAR-2, and VAR-3 contain unique carboxy terminal amino acid sequence disclosed herein as SEQ ID NO: 8, 10 or 12, respectively. Furthermore, a nine amino acid sequence spanning the junction between conserved exon 2 and the newly identified exons present in the particular alternatively spliced EP₄ receptor variant from VAR-1/VAR-3, or VAR-2, are disclosed herein as SEQ ID NO: 14 and SEQ ID NO: 16, respectively. These nine amino acid sequences begin with four amino acid residues that correspond to amino acid sequence present in conserved exon 2 and further include five amino acid residues derived from newly identified exons present in a particular alternatively spliced EP₄ receptor variant. Thus, the invention provides an isolated polypeptide containing the amino acid sequence of SEQ ID NO: 10 or 16. The invention further provides an isolated polypeptide containing an amino acid sequence having at least 50% amino acid identity with SEQ ID NO: 18 and containing the amino acid sequence of SEQ ID NO: 8, 10, 12, 14 or 16, or a conservative variant thereof. As non-limiting examples, the invention provides an isolated polypeptide containing the amino acid sequence of SEQ ID NO: 2, 4 or 6, or a conservative variant thereof, or an isolated polypeptide containing or consisting of the amino acid sequence of SEQ ID NO: 2, 4 or 6.

Further provided herein is an isolated polypeptide containing or consisting of substantially the same amino acid sequence as SEQ ID NO: 2, 4, 6, 8, 10, 12, 14 or 16. The term “substantially the same,” when used herein in reference to an amino acid sequence, means a polypeptide having a similar, non-identical sequence that is considered by those skilled in the art to be a functionally equivalent amino acid sequence. An amino acid sequence that is substantially the same as a reference amino acid sequence can have at least 70%, at least 80%, at least 90%, or at least 95% or more identity to the reference sequence. The term substantially the same amino acid sequence also includes sequences encompassing, for example, modified forms of naturally occurring amino acids such as D-stereoisomers, non-naturally occurring amino acids; amino acid analogs and mimetics, so long as the polypeptide containing such a sequence retains a functional activity of the reference EP₄ receptor variant. A functional activity of an EP₄ receptor variant of the invention can be, for example, the ability to bind a compound such as, but not limited to, PGE₂, an EP₄ receptor antagonist such as ONO-AE2-227, or an EP₄ receptor agonist such as ONO-AE1-329, or the ability to initiate a particular intracellular signal transduction pathway.

It is understood that minor modifications in primary amino acid sequence can result in a polypeptide that has a substantially equivalent function as compared to a polypeptide of the invention. These modifications can be deliberate, as through site-directed mutagenesis, or may be accidental such as through spontaneous mutation. For example, it is understood that only a portion of the entire primary structure of an EP₄ receptor variant can be required in order to bind to compound such as PGE₂, ONO-AE2-227 or ONO-AE1-329. Moreover, functional fragments of an EP₄ receptor variant of the invention containing the amino acid sequence of SEQ ID NO: 2, 4, 6, 8, 10, 12, 14 or 16 similarly are included within the definition of substantially the same amino acid sequence as long as at least one biological function of the EP₄ receptor variant is retained. It is understood that various molecules can be attached to an EP₄ receptor variant or other polypeptide of the invention including, without limitation, heterologous polypeptides, carbohydrates, lipids, and chemical moieties such as radioactive and fluorescent moieties.

The invention further provides an EP₄ receptor variant binding agent which binds a polypeptide of the invention, such as the amino acid sequence of SEQ ID NO: 10, or an epitope thereof. As discussed above, SEQ ID NO: 10 represents the unique carboxy terminal amino acid sequence of alternatively spliced EP₄ receptor variant VAR-2. An EP₄ receptor variant binding agent of the invention can be, without limitation, an antibody or antigen binding fragment thereof which binds the amino acid sequence of SEQ ID NO: 10, or an epitope thereof.

As used herein, the term “EP₄ receptor variant binding agent” means a molecule, such as a simple or complex organic molecule, carbohydrate, peptide, peptidomimetic, protein, glycoprotein, lipoprotein, lipid, nucleic acid molecule, antibody, aptamer or the like that specifically binds the unique carboxy-terminal sequence of an EP₄ receptor variant amino acid sequence disclosed herein. In one embodiment, the EP₄ receptor variant binding agent specifically binds to an amino acid sequence of SEQ ID NO: 10, or an epitope thereof. It is understood that such a binding agent does not specifically bind to a wild-type EP₄ receptor such as SEQ ID NO: 18 since a wild-type EP₄ receptor does not contain the unique carboxy terminal amino acid sequences disclosed herein.

An EP₄ receptor variant binding agent of the invention can be a polypeptide that specifically binds with high affinity or avidity to SEQ ID NO: 10, without substantial cross-reactivity to other unrelated sequences. The affinity of an EP₄ receptor variant binding agent of the invention generally is greater than about 10⁻⁴ M and can be greater than about 10⁻⁶ M, typically being in the range of 10⁻⁴ M to 10⁻¹⁰ M. An EP₄ receptor variant binding agent of the invention can bind, for example, with high affinity such as an affinity of 10⁻⁷ M to 10⁻¹⁰ M. Specific examples of binding agents of the invention include, but are not limited to, polyclonal and monoclonal antibodies that specifically bind an epitope within SEQ ID NO: 10; and nucleic acid molecules, nucleic acid analogs, and small organic molecules, identified, for example, by affinity screening of a nucleic acid or small molecule library against SEQ ID NO: 10. For certain applications, an EP₄ receptor variant binding agent can be utilized that preferentially recognizes a particular conformational or post-translationally modified state of SEQ ID NO: 10. It is understood that an EP₄ receptor variant binding agent of the invention can be labeled with a detectable moiety, if desired, or rendered detectable by specific binding to a detectable secondary agent.

In one embodiment, an EP₄ receptor variant binding agent of the invention is an antibody or antigen-binding fragment thereof. As used herein, the term “antibody” is used in its broadest sense to mean a polyclonal or monoclonal antibody or an antigen binding fragment of such an antibody. Such an antibody of the invention is characterized by having specific binding activity for SEQ ID NO: 10, or an epitope thereof, of at least about 1×10⁵ M⁻¹. Thus, Fab, F(ab′)₂, Fd and Fv fragments of an antibody, which retain specific binding activity for SEQ ID NO: 10, or an epitope thereof, are included within the definition of antibody as used herein. Specific binding activity can be readily determined by one skilled in the art, for example, by comparing the binding activity of the antibody to SEQ ID NO: 10, versus a control sequence. Methods of preparing polyclonal or monoclonal antibodies are well known to those skilled in the art. See, for example, Harlow and Lane, Antibodies: A Laboratory Manual Cold Spring Harbor Laboratory Press (1988).

It is understood that the term antibody includes naturally occurring antibodies as well as non-naturally occurring antibodies such as, without limitation, single chain antibodies, chimeric, bi-functional and humanized antibodies, and antigen-binding fragments thereof. Such non-naturally occurring antibodies can be constructed using solid phase peptide synthesis, produced recombinantly or obtained, for example, by screening combinatorial libraries consisting of variable heavy chains and variable light chains as described in Huse et al., Science 246:1275-1281 (1989). These and other methods of making, for example, chimeric, humanized, CDR-grafted, single chain, and bi-functional antibodies are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); and Borrabeck, Antibody Engineering 2d ed. (Oxford University Press 1995)).

An antibody of the invention can be prepared using as an antigen a polypeptide or peptide containing SEQ ID NO: 10, or an epitope thereof, which can be prepared, for example, from natural sources, produced recombinantly, or chemically synthesized. Such a polypeptide or peptide is a functional antigen if the polypeptide or peptide can be used to generate an antibody that specifically binds SEQ ID NO: 10, or an epitope thereof. As is well known in the art, a non-antigenic or weakly antigenic polypeptide or peptide can be made antigenic by coupling the polypeptide or peptide to a carrier molecule such as bovine serum albumin (BSA) or keyhole limpet hemocyanin (KLH). Various other carrier molecules and methods for coupling a polypeptide or peptide to a carrier molecule are well known in the art (see, for example, Harlow and Lane, supra, 1988). An antigenic polypeptide or peptide can also be generated by expressing the polypeptide or peptide as a fusion protein, for example, fused to glutathione S transferase, polyHis or the like. Methods for expressing polypeptide fusions are well known to those skilled in the art as described, for example, in Ausubel et al., Current Protocols in Molecular Biology (Supplement 47), John Wiley & Sons, New York (1999).

The present invention also provides a cell that includes an exogenously expressed polypeptide containing the amino acid sequence of SEQ ID NO: 10. Further provided herein is a cell that includes an exogenously expressed polypeptide containing an amino acid sequence having at least 50% amino acid identity with SEQ ID NO: 18, and-containing the amino acid sequence of SEQ ID NO: 8, 10, 12, 14 or 16, or a conservative variant thereof. The invention also provides a cell that includes an exogenously expressed polypeptide containing the amino acid sequence of SEQ ID NO: 2, 4 or 6, or a conservative variant thereof. For example, the invention provides a cell that includes an exogenously expressed polypeptide containing or consisting of the amino acid sequence of SEQ ID NO: 2, 4. or 6.

Such a cell containing an exogenously expressed polypeptide of the invention can be generated by expressing a nucleic acid molecule encoding the polypeptide in a suitable host cell, such as a bacterial cell, yeast cell, insect cell, oocyte or other amphibian cell, or mammalian cell, using methods well known in the art. Suitable expression vectors are well known in the art and include vectors in which a nucleic acid molecule is operatively linked to a regulatory element such as a promoter or enhancer region that is capable of regulating expression of a linked nucleic acid molecule. Appropriate expression vectors include, without limitation, those that can be replicated in eukaryotic or prokaryotic cells, those that remain episomal as well as those which integrate into the host cell genome, and those including constitutive, inducible or regulated promoters, enhancers or other regulatory elements.

Suitable expression vectors for prokaryotic or eukaryotic cells are well known to those skilled in the art (see, for example, Ausubel et al., supra, 1999). Eukaryotic expression vectors can contain, for example, a regulatory element such as, but not limited to, the SV40 early promoter, the cytomegalovirus (CMV) promoter, the mouse mammary tumor virus (MMTV) steroid-inducible promoter, the Moloney murine leukemia virus (MMLV) promoter, and the like. One skilled in the art will know or can readily determine an appropriate expression vector for a particular host cell.

Useful expression vectors optionally contain a regulatory element that provides cell or tissue specific expression or inducible expression of the operatively linked nucleic acid molecule. One skilled in the art can readily determine an appropriate tissue-specific promoter or enhancer that allows expression of a polypeptide of the invention in a desired tissue. Furthermore, any of a variety of inducible promoters or enhancers can also be included in an expression vector for regulated expression of a polypeptide of the invention. Such inducible systems include, yet are not limited to, a tetracycline inducible gene regulatory region (Gossen & Bijard, Proc. Natl. Acad. Sci. USA 89:5547-5551 (1992); Gossen et al., Science 268:1766-1769 (1995); Clontech, Palo Alto, Calif.); a metallothionein promoter inducible by heavy metals; an insect steroid hormone responsive gene regulatory region responsive to ecdysone or related steroids such as muristerone (No et al., Proc. Natl. Acad. Sci. USA 93:3346-3351 (1996); Yao et al., Nature 366:476-479 (1993); Invitrogen, Carlsbad, Calif.); a mouse mammary tumor virus (MMTV) gene regulatory region induced by steroids such as glucocortocoid and estrogen (Lee et al., Nature 294:228-232 (1981); and a heat shock promoter.

An expression vector useful in the invention can be a viral vector such as, without limitation, a retrovirus, adenovirus, adeno-associated virus, lentivirus, or herpesvirus vector. Viral based systems provide the advantage of being able to introduce relatively high levels of a heterologous nucleic acid molecule into a variety of cells. Additionally, certain viral vectors can introduce heterologous DNA into non-dividing cells. A variety of suitable viral expression vectors are well known in the art and include, without limitation, herpes simplex virus vectors (U.S. Pat. No. 5,501,979), vaccinia virus vectors (U.S. Pat. No. 5,506,138), cytomegalovirus vectors (U.S. Pat. No. 5,561,063), modified Moloney murine leukemia virus vectors (U.S. Pat. No. 5,693,508), adenovirus vectors (U.S. Pat. Nos. 5,700,470 and 5,731,172), adeno-associated virus vectors (U.S. Pat. No. 5,604,090), constitutive and regulatable retrovirus vectors (U.S. Pat. Nos. 4,405,712; 4,650,764 and 5,739,018, respectively), papilloma virus vectors (U.S. Pat. Nos. 5,674,703 and 5,719,054), and the like.

A cell can be generated that transiently or stably expresses an exogenously expressed polypeptide of the invention. Expression vectors for transient or stable expression of a polypeptide of the invention can be introduced into cells using transfection methods well known to one skilled in the art. Such methods include, without limitation, infection using viral vectors, lipofection, electroporation, particle bombardment and transfection such as calcium-phosphate mediated transfection. Detailed procedures for these methods can be found in Sambrook et al., Molecular Cloning: A Laboratory Manual Cold Spring Harbor Laboratory Press (1989), and the references cited therein. Useful mammalian expression vectors and methods of introducing such vectors into mammalian cells either ex vivo or in vivo are well known in the art. As non-limiting examples, a plasmid expression vector can be introduced into a cell by calcium-phosphate mediated transfection, DEAE dextran-mediated transfection, lipofection, polybrene- or polylysine-mediated transfection, electroporation, or by conjugation to an antibody, gramacidin S, artificial viral envelope or other intracellular carrier. A viral expression vector can be introduced into a cell by infection or transduction, for example, or by encapsulation in a liposome. It further is understood that polypeptides can be delivered directly into cells using a lipid-mediated delivery system (Zelphati et al., J. Biol. Chem. 276:35103-35110 (2001)) to produce a cell that contains an exogenously expressed polypeptide of the invention.

Exemplary host cells that can be used to exogenously express a polypeptide of the invention include, yet are not limited to, primary mammalian cells; established mammalian cell lines such as COS, CHO, HeLa, NIH3T3, HEK 293, and HEK 293/EBNA cells; amphibian cells such as Xenopus embryos and oocytes; and other vertebrate cells. Exemplary host cells further include, without limitation, insect cells such as Drosophila, Spodoptera frugiperda and other cells compatible with baculovirus expression systems (Murakimi et al., Cytokine 13:18-24 (2001); yeast cells such as Saccharomyces cerevisiae, Saccharomyces pombe, or Pichia pastoris; and prokaryotic cells such as Escherichia coli. Following transfection, cells exogenously expressing a polypeptide of the invention can be selected, for example, using drug resistance. A quantitative assay such as, for example, immunoblot analysis, immunoprecipitation or ELISA can determine the amount of a polypeptide of the invention expressed in a transfected cell. Such methods are known to one skilled in the art and can be found, for example, in Ausubel et al., supra, 1989, or in Harlow et al., supra, 1988.

Further provided herein are methods for identifying a compound that modulates an EP₄ receptor variant, identifying a compound that differentially modulates an EP₄ receptor variant, identifying a compound that specifically binds an EP₄ receptor variant, and identifying a compound that differentially binds to an EP₄ receptor variant. In particular, the invention provides a method for identifying a compound that modulates an EP₄ receptor variant by contacting an EP₄ receptor variant with a compound and determining the level of an indicator which correlates with modulation of the EP₄ receptor variant, where an alteration in the level of the indicator as compared to a control level indicates that the compound is a compound that modulates the EP₄ receptor variant. Further provided herein are methods for identifying a compound that modulates an EP₄ receptor variant by contacting an isolated EP₄ receptor variant or an EP₄ receptor variant over-expressed in a genetically engineered cell with a compound and determining the level of an indicator which correlates with modulation of the EP₄ receptor variant, where an alteration in the level of the indicator as compared to a control level indicates that the compound is a compound that modulates the EP₄ receptor variant.

As used herein in reference to an EP₄ receptor variant, the term “modulates” means the ability to alter a characteristic of an EP₄ receptor variant. A characteristic of an EP₄ receptor variant that can be altered can be, without limitation, an amount, activity, or physical conformation of an EP₄ receptor variant. As a non-limiting example, a compound that modulates an EP₄ receptor variant can increase or decrease the binding of an EP₄ receptor variant to a ligand such as PGE₂, or another EP₄ receptor agonist or antagonist such as ONO-AE1-329 or ONO-AE2-227. Also, for example, a compound can increase or decrease the binding of an EP₄ receptor variant to an intracellular signaling molecule that initiates a signal transduction pathway within a cell. It is understood that compounds that modulate an EP₄ receptor variant include compounds that specifically bind to an EP₄ receptor variant as well as compounds that do not specifically bind to an EP₄ receptor variant.

A method of the invention for identifying a compound that modulates an EP₄ receptor variant involves determining the level of an indicator which correlates with modulation of the EP₄ receptor variant, where an alteration in the level of the indicator as compared to a control level indicates that the compound modulates the EP₄ receptor variant. As used herein, the term “indicator” means a detectable substance which is altered qualitatively or quantitatively in response to modulation of an EP₄ receptor variant. An indicator can be a substance that is normally present in a cell such as a signal transduction molecule, or a substance that is exogenously expressed or otherwise added to a cell, the level of which correlates with modulation of an EP₄ receptor variant. One example of an indicator is luciferase. Examples of signal transduction molecules which can act as indicators include, without limitation, the intracellular substances cyclic AMP, inositol phosphates and calcium, the level of which can be altered in response to modulation of an EP₄ receptor variant.

As understood by those of skill in the art, assay methods for identifying compounds that modulate an EP₄ receptor variant generally involve comparison to a control. For example, in a method of the invention, an alteration in the level of an indicator which correlates with modulation of an EP₄ receptor variant is compared to a control level of the indicator. One type of a control is a sample that is treated substantially the same as the EP₄ receptor variant which is contacted with a compound, with the distinction that the control sample is not exposed to the compound. Controls include, but are not limited to, historical reference values, and samples that are assayed simultaneously or sequentially in comparison to the EP₄ receptor variant which is contacted with the compound.

In one embodiment, a method of the invention is practiced using calcium as the indicator. For example, as disclosed herein in Example III, a FLIPR assay can be used to identify compounds that modulate an EP₄ receptor variant by determining the level of calcium that results after contacting the variant with a compound. One skilled in the art understands that exogenously expressed reporter molecules such as, for example, luciferase, β-galactosidase and green fluorescent protein (GFP) also can be indicators useful in a method of the invention (see Example III).

Further provided herein are methods for identifying a compound that specifically binds to an EP₄ receptor variant by contacting an EP₄ receptor variant with a compound and determining specific binding of the compound to the EP₄ receptor variant. Additionally provided herein are methods for identifying a compound that specifically binds to an EP₄ receptor variant by contacting an isolated EP₄ receptor variant or an EP₄ receptor variant over-expressed in a genetically engineered cell with a compound and determining specific binding of the compound to the EP₄ receptor variant.

As used herein in reference to a compound and an EP₄ receptor variant, the term “specific binding” means binding with an affinity for the target EP₄ receptor variant that is measurably higher than the affinity for an unrelated polypeptide such as an unrelated G protein coupled receptor, for example, a rhodopsin receptor. Thus, a polypeptide or small molecule compound that specifically binds an EP₄ receptor variant has an affinity for the EP₄ receptor variant that is measurably higher than its affinity for an unrelated polypeptide. Binding affinity can be low or high so long as the binding is sufficient to be detectable. For example, a compound can specifically bind an EP₄ receptor variant with a binding affinity (Kd) of about 10⁻⁴ M or less, 10⁻⁵ M or less, 10⁻⁶ M or less, 10⁻⁷ M or less, 10⁻⁸ M or less, or 10⁻⁹ M or less. Several methods for detecting or measuring specific binding are well known in the art and discussed further below.

The screening methods of the invention can be practiced using, for example, using an EP₄ receptor variant over-expressed in a genetically engineered cell. As used herein, the term “genetically engineered cell” means a cell having genetic material which is altered by the hand of man. Such a cell can contain a transient or permanent alteration of its genetic material including, for example, alteration in genomic or episomal genetic material. The genetic material in a genetically engineered cell can be altered using, without limitation, an exogenously expressed nucleic acid molecule, chemical mutagen or transposable element. It is understood that a genetically engineered cell can contain one or more man-made alterations; for example, a cell can be co-transfected with more than one expression vector. As used herein in relation to an EP₄ receptor variant in a genetically engineered cell, the term “over-expressed” means having a protein level of an EP₄ receptor variant greater than the level seen in a corresponding non-genetically engineered cell.

As understood by one skilled in the art, an EP₄ receptor variant can be over-expressed in a genetically engineered cell, for example, by exogenously expressing a nucleic acid molecule encoding the EP₄ receptor variant in a cell as described herein above. It is further understood that an EP₄ receptor variant can be over-expressed in a cell that does not normally express the EP₄ receptor variant, or in a cell that naturally expresses the endogenous EP₄ receptor variant. As a non-limiting example, an EP₄ receptor variant can be over-expressed in a cell that expresses endogenous EP₄ receptor variant at a low level. In addition, an EP₄ receptor variant can be over-expressed in a genetically engineered cell, for example, by expressing a regulatory molecule in the cell to increase expression of the endogenous EP₄ receptor variant. Another example of a method whereby an EP₄ receptor variant can be over-expressed in a genetically engineered cell is recombination of a heterologous regulatory region such as, without limitation, a promoter, enhancer or 3′ regulator, in the cell such that the heterologous regulatory region results in over-expression of endogenous EP₄ receptor variant. As understood by one skilled in the art, over-expression of an EP₄ receptor variant in a genetically engineered cell includes, without limitation, over-expression of the variant on the surface of the cell, within a cell membrane or in the cytosolic portion of the cell.

An EP₄ receptor variant also can be over-expressed in a cell using a chemical agent. Thus, the invention provides a method for identifying a compound that modulates an EP₄ receptor variant by contacting the EP₄ receptor variant with a compound, where the EP₄ receptor variant is over-expressed in a cell using a chemical agent, and determining the level of an indicator which correlates with modulation of the EP₄ receptor variant, where an alteration in the level of the indicator as compared to a control level indicates that the compound is a compound that modulates the EP₄ receptor variant. The invention also provides a method for identifying a compound that specifically binds to an EP₄ receptor variant by contacting the EP₄ receptor variant with a compound, where the EP₄ receptor variant is over-expressed in a cell using a chemical agent, and determining specific binding of the compound to the EP₄ receptor variant. Chemical agents that can result in over-expression of an EP₄ receptor variant can include, without limitation, chemicals that induce the level or activity of a regulatory factor, such as a transcription factor, that promotes EP₄ receptor variant expression; and chemicals that suppress the level or activity of a regulatory factor, such as a transcription factor, that inhibits EP₄ receptor variant expression.

As described above, the methods of the invention can be practiced with a cell that over-expresses an EP₄ receptor variant. In addition, it is understood that cellular extracts prepared from cells that over-express an EP₄ receptor variant such as genetically engineered cells that over-express an EP₄ receptor variant can be useful in the methods of the invention. Methods for generating different types of cellular extracts including, without limitation, whole cell extracts, membrane extracts, cytosolic extracts and nuclear extracts are well known in the art. As a non-limiting example, receptor enriched plasma membrane fractions can be obtained by continuous or discontinuous gradients of, for example, sucrose, as described in Woodward and Lawrence, Biochemical Pharmacology 47:1567-1674 (1994).

Isolated EP₄ receptor variants also can be useful in the screening methods of the invention. As used herein in reference to an EP₄ receptor variant, the term “isolated” means an EP₄ receptor variant which is substantially separated from other polypeptides. For example, an isolated EP₄ receptor variant derived from a cell can be substantially purified away from other polypeptides in the cell. An isolated EP₄ receptor variant can contain non-polypeptide components, for example, an isolated EP₄ receptor variant can be associated with a natural or artificial lipid containing membrane. In one embodiment, a method of the invention is practiced with an isolated EP₄ receptor variant that contains an amino acid sequence having at least 50% amino acid identity with SEQ ID NO: 18 and containing the amino acid sequence of SEQ ID NOS: 8, 10, 12, 14 or 16, or a conservative variant thereof. In another embodiment, a method of the invention is practiced with an EP₄ receptor variant that contains the amino acid sequence of SEQ ID NOS: 2, 4 or 6, or a conservative variant thereof. In a further embodiment, a method of the invention is practiced with an isolated EP₄ receptor variant that contains or consists of the amino acid sequence of SEQ ID NO: 2, 4 or 6.

An EP₄ receptor variant of the invention can be prepared in isolated form using conventional biochemical purification methods, starting either from a tissue containing the desired EP₄ receptor variant or from a recombinant source. An EP₄ receptor variant can be isolated by any of a variety of methods well-known in the art, including, but not limited to, precipitation, gel filtration, ion-exchange, reverse-phase and affinity chromatography, and combinations thereof. Other well-known methods for protein isolation are described in Deutscher et al., Guide to Protein Purification: Methods in Enzymology Vol. 182, (Academic Press, (1990)). Methods suitable for isolating an EP₄ receptor variant using biochemical purification are known in the art as described for example, in Venter and Harrison, (eds), Receptor Purification Procedures (Liss, (1984)); Litwack, Receptor Purification: Receptors for CNS Agents, Growth Factors Hormones, & Related Substances (Humana Press, (1990)); or Litwack, Receptor Purification: Receptors for Steroid Hormones, Thyroid Hormones, Water Balancing Hormone, & Others (Humana Press, (1990)). Purification of the receptor variant can be routinely monitored, for example, by an immunological assay or functional assay such as a ligand binding assay.

An isolated EP₄ receptor variant of the invention also can be produced by chemical synthesis. As a non-limiting example, synthetic isolated EP₄ receptor variants, including fragments thereof, can be produced using an Applied Biosystems, Inc., Model 430A or 431A automatic peptide synthesizer employing the chemistry provided by the manufacturer (Applied BioSystems, Inc.; Foster City, Calif.). Methods for synthesizing isolated polypeptides are well known in the art. See, for example, Bodanzsky, Principles of Peptide Synthesis (1st ed. & 2d rev. ed.), Springer-Verlag, New York, New York (1984 & 1993), Chapter 7; and Stewart and Young, Solid Phase Peptide Synthesis (2d ed.), Pierce Chemical Co., Rockford, Ill. (1984).

In the methods of the invention for identifying a compound that modulates, or specifically binds to, an EP₄ receptor variant, an isolated EP₄ receptor variant or EP₄ receptor variant over-expressed in a genetically engineered cell can be contacted with a compound in a solution under conditions suitable for interaction between the EP₄ receptor variant and compound. Such contact can occur in an isolated cell in cell culture, in a whole or partially purified cell extract, or with an isolated polypeptide. As used herein, the term “in vitro” means in an artificial environment outside of a living organism or cell. Assays performed in a test tube, microcentrifuge tube, 96 well plate, 384 well plate, 1536 well plate or other assay format outside of an organism or living cell are in vitro assays. Experiments performed in cells or tissues that have been fixed and are therefore dead (sometimes referred to as in situ experiments) or using cell-free extracts from cells are in vitro. Contact can also occur in vivo using, for example, living primary or tissue culture cells, isolated tissues or organs, or whole animals.

Conditions suitable for contacting an isolated EP₄ receptor variant or EP₄ receptor variant over-expressed in a genetically engineered cell with a compound are dependent on the characteristics of the EP₄ receptor variant and the compound. For example, the overall charge of the EP₄ receptor variant and the compound can be considered when adjusting the salt concentration or pH of a buffering solution to optimize the specific binding or modulation of the EP₄ receptor variant by the compound. Usually a salt concentration and pH in the physiological range, for example, about 100 mM KCl and pH 7.0 are reasonable starting points. In addition, other components such as glycerol or protease inhibitors can be added to the solution, for example, to inhibit polypeptide degradation. It is understood that the stability of the contact between the EP₄ receptor variant and the compound can be effected by the temperature at which such contact occurs and that the optimal temperature for contact can be routinely determined by those skilled in the art. For example, reactions can be performed on ice (4° C.), at room temperature (about 25° C.) or at body temperature (37° C.). Suitable conditions can be similar or identical to conditions used for binding of a compound to the wild-type human EP₄ receptor. Such conditions are known in the art and include, for example, contact in a binding buffer containing 10 mM MES/KOH (pH 6.0), 1 mM EDTA, and 10 mM MnCl₂, and incubation at 30° C. for 45 minutes, as described in Bastien et al., J. Biol. Chem. 269:11873-11877 (1994).

The screening methods of the invention are useful for identifying compounds that modulate or differentially modulate, or that specifically or differentially bind an EP₄ receptor variant. As used herein, the term “compound” means a molecule of natural or synthetic origin. A compound can be, without limitation, a small organic or inorganic molecule, polypeptide, peptide, peptidomimetic, non-peptidyl compound, carbohydrate, lipid, antibody or antibody fragment, aptamer, or nucleic acid molecule. In one embodiment, the compound is a small organic molecule. It is understood that a compound can have a known or unknown structure, and can be assayed as an isolated molecule or as part of a population of compounds.

As understood by one skilled in the art, a compound can specifically bind to an EP₄ receptor variant without modulating the EP₄ receptor variant; specifically bind to an EP₄ receptor variant, thereby modulating the EP₄ receptor variant; or modulate an EP₄ receptor variant without specifically binding the EP₄ receptor variant. Compounds that specifically bind to an EP₄ receptor variant can include, without limitation, PGE₂ and prostanoid-like compounds. A compound that modulates an EP₄ receptor variant but does not directly bind to the EP₄ receptor variant can be, for example, a compound that binds to or effects the activity of a polypeptide in a cell, where that polypeptide increases or decreases the level of an EP₄ receptor variant. Such polypeptides include, without limitation, transcription or translation regulatory factors, signal transduction polypeptides; kinases and phosphatases; polypeptides that bind to an EP₄ receptor variant; and anti-sense oligonucleotides, inhibitor RNA molecules and ribozymes, which act on the nucleic acid that encodes the EP₄ receptor variant.

Compounds that modulate or specifically bind to an EP₄ receptor variant further include, but are not limited to, agonists and antagonists. An agonist is a compound that binds to a receptor and activates it, producing a pharmacological response such as contraction, relaxation, secretion, or enzyme activation. An antagonist is a compound which attenuates the effect of an agonist. An antagonist can be competitive, meaning it binds reversibly to a region of the receptor in common with an agonist, but occupies the site without activating the effector mechanism. The effects of a competitive antagonist can be overcome by increasing the concentration of agonist, thereby shifting the equilibrium and increasing the proportion of receptors occupied by agonist. Alternatively, antagonists can be non-competitive, where no amount of agonist can completely overcome the inhibition once it has been established. Non-competitive antagonists can bind covalently to the agonist binding site (called competitive irreversible antagonists), in which case there is a period before the covalent bond forms during which competing ligands can prevent the inhibition. Other types of non-competitive antagonists act allosterically at a different site on the receptor.

Other classes of compounds that can modulate or specifically bind to an EP₄ receptor variant include inverse agonists, which are compounds which produce an opposite physiological effect to that of an agonist, yet act at the same receptor. Such compounds have also been described as negative antagonists, or as having negative efficacy. Another class of compounds that can modulate or specifically bind to an EP₄ receptor variant is partial agonists, which are agonists that are unable to produce maximal activation of the receptor.

A library of compounds can be useful in the screening methods of the invention. Such a library can be a random collection of compounds or a focused collection of compounds, for example, compounds that are rationally designed or pre-selected based on a particular physical or functional characteristic. As a non-limiting example, a library of prostanoids or prostanoid-related compounds can be useful in the screening methods of the invention. Libraries useful in the methods of the invention include, yet are not limited to, natural product libraries derived from, without limitation, microorganisms, animals, plants, and marine-organisms; combinatorial chemical or other chemical libraries such as those containing randomly synthesized compounds; combinatorial libraries containing structural analogs of prostanoids or other known compounds, or random or biased assortments of, for example, small organic molecules, polypeptides, oligonucleotides, and combinations thereof. Still other libraries of interest include peptidomimetic libraries, multiparallel synthetic collections, and recombinatorial libraries. Combinatorial and other chemical libraries are known in the art, as described, for example, in Myers, Curr. Opin. Biotechnol. 8:701-707 (1997). Appropriate libraries also can be assembled from catalog sources such as Cayman Chemical Co. (Ann Arbor, Mich.), BIOMOL Research Laboratories, Inc. (Plymouth Meeting, Pa.), Tocris Cooksoon Inc. (Ellisville, Mo.), and others. These libraries can include, without limitation, fatty acids, fatty acid amides and esters, and eicosanoids.

In a screening method of the invention, the members of a library of compounds can be assayed for activity individually, in pools, or en masse. An example of en masse screening to identify a compound that modulates or specifically binds to an EP₄ receptor variant is as follows: a library of compounds is assayed in pools for the ability to modulate or specifically bind an EP₄ receptor variant; the sub-population which modulates or specifically binds the EP₄ receptor variant is subdivided; and the assay is repeated as needed in order to isolate an individual compound or compounds from the library that modulate or specifically bind the EP₄ receptor variant.

The methods of the invention can utilize high throughput screening (HTS) techniques to identify compounds that modulate or specifically bind to an EP₄ receptor variant. HTS assays permit screening of large numbers of compounds in an efficient manner. Cell-based high throughput screening systems include, but are not limited to, melanophore assays, yeast-based assay systems, and mammalian cell expression systems (Jayawickreme and Kost, Curr. Opin. Biotechnol. 8:629-634 (1997)). Automated and miniaturized high throughput screening assays are also useful in the methods of the invention (Houston and Banks, Curr. Opin. Biotechnol. 8:734-740 (1997)). High throughput screening assays are designed to identify “hits” or “lead compounds” having the desired modulating or specific binding activity, from which modified compounds can be prepared to improve a property of the initial lead compound. Chemical modification of the “hit” or “lead compound” can be based on an identifiable structure/activity relationship (SAR) between the “hit” and an EP₄ receptor variant of the invention. It is understood that assays including, but not limited to, the melanophore and radioligand binding assays disclosed below, and the FLIPR and luciferase assays disclosed in Example III, can be performed as conventional or high through-put screening assays to identify a compound that modulates or specifically binds to an EP₄ receptor variant according to a method of the invention.

Various types of assays can be useful for identifying a compound that modulates or specifically binds to an EP₄ receptor variant in a method of the invention. For example, any of various assays can be used to measure specific binding of a compound to an EP₄ receptor variant in a method of the invention. A classic assay used for measuring specific binding of a compound to a receptor is a radioligand binding assay. Radioligand binding assays can be performed on cells or in solution, for example, using isolated cell membranes. As a non-limiting example, cells or cell membranes that transiently or stably over-express an EP₄ receptor variant can be incubated with a ligand such as a novel or known ligand, for example, radioactively labeled PGE₂. After washing away any unbound radioactively labeled PGE₂, compounds of interest can be incubated with the cells. After incubation, the solution around the cells is collected, and the amount of radioactively labeled PGE₂ in the solution is determined using, for example, a scintillation counter. Compounds that specifically bind to the EP₄ receptor variant displace radioactively labeled PGE₂ from the receptor and thereby increase radioactively labeled. PGE₂ in the solution. A method for a whole cell radioligand binding assay using PGE₂ is described, for example, in Bastien et al., supra, 1994. As understood by one skilled in the art, a ligand such as PGE₂ also can be labeled with a non-radioactive moiety such as a fluorescent moiety in a similar binding assay.

A variety of other assays well known in the art can be used to determine specific binding of a compound to an EP₄ receptor variant in a method of the invention. Such assays include, without limitation, detecting specific binding of a labeled compound to an EP₄ receptor variant which is immobilized. For example, a compound can be conjugated to a radiolabel, fluorescent label or enzyme label such as alkaline phosphatase, horse radish peroxidase or luciferase. Labeled compound can then bind to an EP₄ receptor variant, for example an EP₄ receptor variant membrane preparation, which is immobilized, for example, on a solid support such as a latex bead. Unbound compound can be washed away, and the amount of specifically bound compound can be detected based on its label. Fluorescently labeled compound can also be bound to an EP₄ receptor variant in solution and bound complexes detected, for example, using a fluorescence polarization assay (Degterev et al., Nature Cell Biology 3:173-182 (2001)). Such assays also can be performed where the EP₄ receptor variant is labeled and the compound is immobilized or in solution. One skilled in the art understands that a variety of additional means can be used to determine specific binding to an EP₄ receptor variant; as non-limiting examples, binding of a compound to a ¹⁵N-labeled EP₄ receptor variant can be detected using nuclear magnetic resonance (NMR), or specific binding can be determined using an antibody that specifically recognizes a ligand-bound EP₄ receptor variant.

High-throughput assays for determining specific binding to an EP₄ receptor variant further include, but are not limited to, scintillation proximity assays (Alouani, Methods Mol. Biol. 138:135-41 (2000)). Scintillation proximity assays involve the use of a fluomicrosphere coated with an acceptor molecule, such as an antibody, to which an antigen will bind selectively in a reversible manner. For example, a compound can be bound to a fluomicrosphere using an antibody that specifically binds to the compound, and contacted with a labeled EP₄ receptor variant. If the labeled EP₄ receptor variant specifically binds to the compound, the radiation energy from the labeled EP₄ receptor variant is absorbed by the fluomicrosphere, thereby producing light which is easily measured. Variations of such assays can also be performed where the EP₄ receptor variant is bound to the fluomicrosphere, and the compound is labeled.

Additional assays suitable for determining specific binding of a compound to an EP₄ receptor variant in a screening method of the invention include, without limitation, UV and chemical cross-linking assays (Fancy, Curr. Opin. Chem. Biol. 4:28-33 (2000)) and biomolecular interaction analyses (Weinberger et al., Pharmacogenomics 1:395-416 (2000)). Specific binding of a compound to an EP₄ receptor variant can be determined by cross-linking these two components, if they are in contact with each other, using UV or a chemical cross-linking agent. In addition, a biomolecular interaction analysis (BIA) can detect whether two components are in contact with each other. In such an assay, one component, such as an EP₄ receptor variant (for example, a membrane preparation containing an EP₄ receptor variant) is bound to a BIA chip, and a second component such as a compound is passed over the chip. If the two components specifically bind, the contact results in an electrical signal, which is readily detected.

In addition, virtual computational methods and the like can be used to identify compounds that modulate or specifically bind to an EP₄ receptor variant in a screening method of the invention. Exemplary virtual computational methodology involves virtual docking of small-molecule compounds on a virtual representation of an EP₄ receptor variant structure in order to determine or predict specific binding. See, for example, Lengauer et al., Current Opinions in Structural Biology 6:402-406 (1996); Choichet et al., Journal of Molecular Biology 221:327-346 (1991); Cherfils et al., Proteins 11:271-280 (1991); Palma et al., Proteins 39:372-384 (2000); Eckert et al., Cell 99:103-115 (1999); Loo et al., Med. Res. Rev. 19:307-319 (1999); and Kramer et al., J. Biol. Chem. (2000).

One type of assay that does not directly measure binding to an EP₄ receptor variant, but measures activation of a signal transduction pathway, is an assay based on melanophores, which are skin cells that provide pigmentation to an organism (Lerner, Trends Neurosci. 17:142-146 (1994)). In numerous animals, including fish, lizards and amphibians, melanophores are used, for example, for camouflage. The color of the melanophore is dependent on the intracellular position of melanin-containing organelles, termed melanosomes. Melanosomes move along a microtubule network and are clustered to give a light color or dispersed to give a dark color. The distribution of melanosomes is regulated by G protein coupled receptors and cellular signaling events, where increased concentrations of second messengers such as cyclic AMP and diacylglycerol result in melanosome dispersion and darkening of melanophores. Conversely, decreased concentrations of cyclic AMP and diacylglycerol result in melanosome aggregation and lightening of melanophores.

A melanophore-based assay can be advantageously used to identify a compound that modulates or specifically binds to an EP₄ receptor variant, due to the regulation of melanosome distribution by EP₄ receptor variant-stimulated intracellular signaling. For example, an EP₄ receptor variant can be over-expressed in genetically engineered melanophore cells, for example, frog melanophore cells. Compounds that modulate or specifically bind to the EP₄ receptor variant can stimulate or inhibit G protein coupled receptor signaling. Both stimulation or inhibition of signaling can be determined since the system can be used to detect both aggregation of melanosomes and lightening of cells, and dispersion of melanosomes and darkening of cells. Thus, the color of the cells, determined by the level of melanin in the cells, is an indicator that can be used to identify a compound that modulates or specifically binds to an EP₄ receptor variant in a method of the invention.

In addition to the methods described above for identifying a compound that modulates or specifically binds an EP₄ receptor variant, the invention also provides related methods for identifying a compound that differentially modulates or differentially binds to an EP₄ receptor variant. It is understood that the EP₄ receptor variants, cells, compounds, indicators, conditions for contacting, and assays described above also can be applied to methods for identifying a compound that differentially modulates or differentially binds to an EP₄ receptor variant.

In particular, the invention provides a method for identifying a compound that differentially modulates an EP₄ receptor variant by a) contacting an isolated EP₄ receptor variant or an EP₄ receptor variant over-expressed in a genetically engineered cell with a compound; b) determining the level of an indicator which correlates with modulation of the EP₄ receptor variant; c) contacting a second receptor with the compound; d) determining the level of a corresponding indicator after contacting of the compound to the second receptor; and e) comparing the level of the indicator from step (b) with the level of the corresponding indicator from step (d), where a different level of the indicator from step (b) compared to the level of the corresponding indicator from step (d) indicates that the compound is a compound that differentially modulates the EP₄ receptor variant.

As described above, an indicator is a detectable substance which is altered qualitatively or quantitatively in response to modulation of an EP₄ receptor variant. A “corresponding indicator” is an indicator that can be compared to the indicator which correlates with modulation of the EP₄ receptor variant in step (b). For example, a corresponding indicator can be the same indicator as the indicator which correlates with modulation of the EP₄ receptor variant in step (b). In addition, for example, a corresponding indicator can be a different indicator as the indicator which correlates with modulation of the EP₄ receptor variant in step (b) so long as the corresponding indicator can be compared to the indicator which correlates with modulation of the EP₄ receptor variant in step (b). As a non-limiting example, the indicator in step (b) can be calcium, and the corresponding indicator can be a substance whose amount is directly correlated with calcium level, such as a signal transduction molecule. As a further non-limiting example, the indicator in step (b) and corresponding indicator in step (d) can be related molecules, such as two different fluorophores. In one embodiment, the level of the indicator which correlates with modulation of the EP₄ receptor variant in step (b) is greater than the level of the corresponding indicator from step (d). In another embodiment, the level of the indicator which correlates with modulation of the EP₄ receptor variant in step (b) is less than the level of the corresponding indicator from step (d).

The invention also provides a method for identifying a compound that differentially binds to an EP₄ receptor variant by a) contacting an isolated EP₄ receptor variant or an EP₄ receptor variant over-expressed in a genetically engineered cell with a compound; b) determining specific binding of the compound to the EP₄ receptor variant; c) contacting a second receptor with the compound; d) determining specific binding of the compound to the second receptor; and e) comparing the level of specific binding from step (b) with the level of specific binding from step (d), where a different level of specific binding from step (b) compared to the level of specific binding from step (d) indicates that the compound is a compound that differentially binds to the EP₄ receptor variant. In one embodiment, the different level of specific binding is an increased level of binding. In another embodiment, the different level of specific binding is a decreased level of binding.

As set forth above in regard to methods for identifying a compound that modulates or specifically binds an EP₄ receptor variant, the EP₄ receptor variant can be any of a variety of EP₄ receptor variants such as an isolated polypeptide containing an amino acid sequence having at least 50% amino acid identity with SEQ ID NO: 18 and containing the amino acid sequence of SEQ ID NO: 8, 10, 12, 14 or 16, or a conservative variant thereof; or an isolated polypeptide containing the amino acid sequence of SEQ ID NO: 2, 4 or 6, or a conservative variant thereof. In one embodiment, the EP₄ receptor variant is over-expressed in a genetically engineered cell. In another embodiment, the EP₄ receptor variant is exogenously over-expressed in a genetically engineered cell.

In the methods of the invention for identifying a compound that differentially modulates or differentially binds an EP₄ receptor variant, the second receptor can be any receptor of interest. For example, the second receptor can be a G-protein coupled receptor such as, without limitation, any other EP₄ receptor such as a different EP₄ receptor variant or a wild-type EP₄ receptor. In particular embodiments, the second receptor is a wild-type EP₄ receptor containing the amino acid sequence SEQ ID NO: 18, or a functional fragment thereof. The second receptor can be, for example, expressed in a cell endogenously or exogenously or can be in isolated form.

It is understood that the methods of the invention can be practiced using an EP₄ receptor variant and second receptor expressed, for example, in different cells. In addition, the methods of the invention can be practiced using an EP₄ receptor variant and second receptor expressed in the same cell, for example, where the EP₄ receptor variant has distinct binding specificity or signal transduction effects as compared to the co-expressed second receptor.

The invention further provides an isolated nucleic acid molecule having a nucleotide sequence that encodes a polypeptide containing the amino acid sequence of SEQ ID NO: 10 or 16. The invention also provides an isolated nucleic acid molecule having a nucleotide sequence that encodes a polypeptide containing an amino acid sequence having at least 50% amino acid identity with SEQ ID NO: 18 and containing the amino acid sequence of SEQ ID NO: 8, 10, 12, 14 or 16, or a conservative variant thereof. The invention further provides an isolated nucleic acid molecule containing a nucleotide sequence that encodes the amino acid sequence of SEQ ID NO: 2, 4 or 6, or a conservative variant thereof. For example, the invention provides an isolated nucleic acid molecule containing the nucleotide sequence of SEQ ID NO: 1, 3 or 5. The invention further provides a vector containing a nucleic acid molecule having a nucleotide sequence that encodes a polypeptide containing an amino acid sequence having at least 50% amino acid identity with SEQ ID NO: 18 and containing the amino acid sequence of SEQ ID NO: 8, 10, 12, 14 or 16, or a conservative variant thereof; or a nucleic acid molecule containing a nucleotide sequence that encodes the amino acid sequence of SEQ ID NO: 2, 4 or 6, or a conservative variant thereof.

Isolated nucleic acid molecules include DNA and RNA molecules as well as both sense or complementary anti-sense strands. It is understood that an isolated nucleic acid molecule of the invention can be a double-stranded or single-stranded molecule, an RNA or DNA molecule, and can optionally include non-coding sequence. Nucleic acid molecules of the invention further include molecules which are wholly or partially chemically synthesized.

The nucleic acid molecules of the invention optionally include heterologous nucleic acid sequences that are not part of the EP₄ receptor variant-encoding sequences in nature. Such a heterologous nucleic acid sequence can be optionally separated from the EP₄ receptor variant-encoding sequence by an encoded cleavage site that facilitates removal of non-EP₄ receptor variant polypeptide sequences from the expressed fusion protein. Heterologous nucleic acid sequences include, without limitation, sequences encoding poly-histidine sequences, FLAG tags and other epitopes, and glutathione-S-transferase, thioredoxin, and maltose binding protein domains or other domains or sequences that facilitate purification or detection of the fusion protein containing an EP₄ receptor variant of the invention.

An isolated nucleic acid molecule of the invention contains nucleotide sequence that is distinct from the nucleotide sequence of the human EP₄ receptor genomic clone AC093264.2 since, for example, the nucleotide sequence which encodes the amino acid sequence SEQ ID NO: 14 or 16 spans the junction between conserved exon 2 and the newly identified alternatively spliced exons. The intron/exon structure of the human EP₄ receptor genomic clone AC093264.2 is shown in FIG. 5.

The location of exons from the human EP₄ receptor genomic clone AC093264.2 that are present in alternatively spliced human EP₄ receptor variants VAR-1 to VAR-3 as determined using BLAST searches are as follows: EP₄ receptor variant VAR-1 alternatively spliced sequence corresponds to human genomic clone AC093264.2 at a range from +65500 to +65406; EP₄ receptor variant VAR-2 alternatively spliced sequence corresponds to human genomic clone AC093264.2 at a range from +65160 to +65100; and EP₄ receptor variant VAR-3 alternatively spliced sequence corresponds to human genomic clone AC093264.2 at a range from +65500 to +65257 and +65160 to +65100.

The invention further provides a vector containing a nucleic acid molecule having a nucleotide sequence that encodes a polypeptide containing an amino acid sequence having at least 50% amino acid identity with SEQ ID NO: 18 and the amino acid sequence of SEQ ID NO: 8, 10, 12, 14 or 16, or a conservative variant thereof. The invention also provides a vector containing a nucleic acid molecule containing a nucleotide sequence that encodes the amino acid sequence of SEQ ID NO: 2, 4, or 6, or a conservative variant thereof. For example, such a vector can contain or consist of a nucleotide sequence that encodes the amino acid sequence of SEQ ID NO: 2, 4, or 6, or contain or consist of a nucleic acid molecule having the nucleotide sequence of SEQ ID NO: 1, 3 or 5. The invention further provides a host cell that includes a vector of the invention.

Vectors are useful, for example, for subcloning and amplifying a nucleic acid molecule encoding a polypeptide of the invention and for recombinantly expressing the encoded EP₄ variant receptor or other polypeptide. Vectors of the invention include, without limitation, viral vectors such as bacteriophage, baculovirus and retrovirus vectors; cosmids and plasmids; and, particularly for cloning large nucleic acid molecules, bacterial artificial chromosome vectors (BACs) and yeast artificial chromosome vectors (YACs). Such vectors are commercially available, and their uses are well known in the art. Vectors further encompass expression vectors such as those discussed herein above.

The invention also provides an isolated nucleic acid molecule containing a nucleotide sequence that encodes the amino acid sequence of SEQ ID NO: 2, 4, 6, 8, 10, 12, 14 or 16. Such a nucleic acid molecule of the invention can be used, without limitation, in recombinant cloning methods or as a nucleic acid probe.

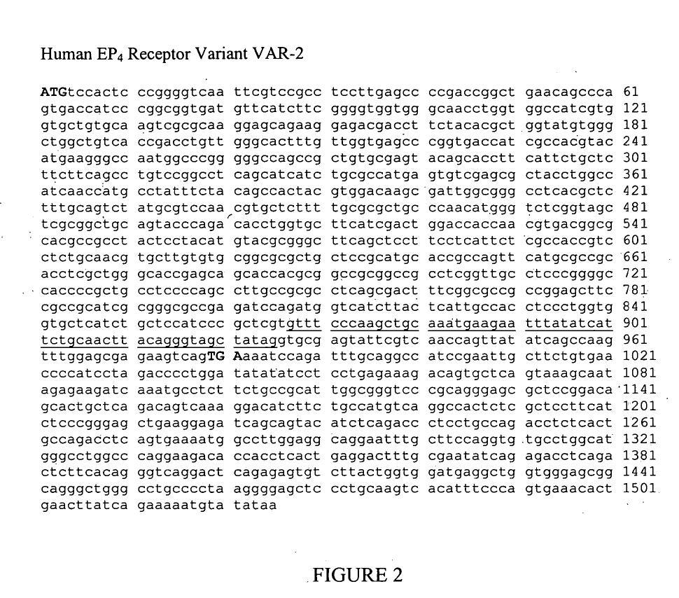

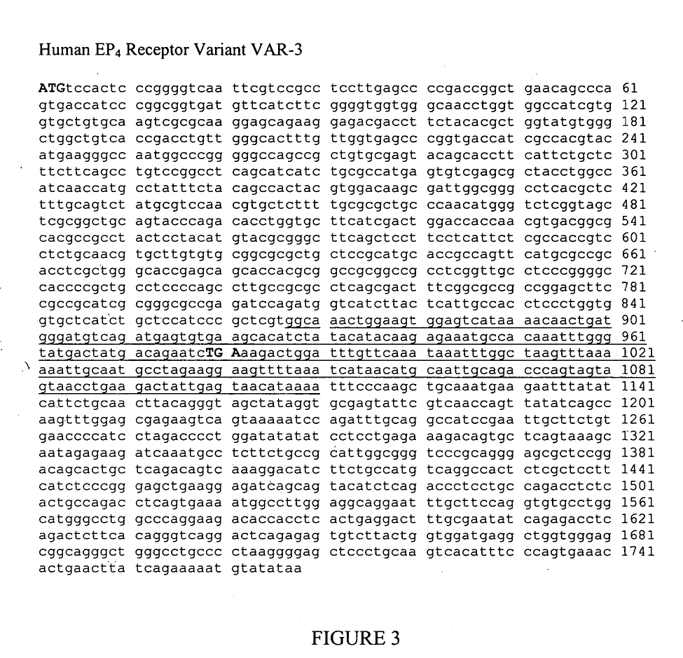

As non-limiting examples, nucleic acid molecules of the invention can be derived from the unique nucleotide sequence which surrounds the junction between conserved exon 2 and the newly identified alternatively spliced exon A, B or C present in the disclosed EP₄ receptor variants. For example, nucleic acid molecules containing 20 nucleotides spanning the splice junction of EP₄ receptor variants include, without limitation, the nucleic acid sequence tcccgctcgtgtttcccaag (SEQ ID NO: 21) at the 5′ splice junction of VAR-2, the nucleic acid sequence gtagctataggtgcgagtat (SEQ ID NO: 22) at the 3′ splice junction of VAR-2, and taacataaaa tttcccaagc (SEQ ID NO: 23) at the 3′ splice junction of VAR-3 (see FIGS. 2 and 3).

As understood by one skilled in the art, a nucleic acid molecule of the invention can contain nucleotide sequence in addition to the nucleotide sequence of SEQ ID NOS: 21, 22, or 23. For example, a nucleic acid molecule of the invention can contain further naturally occurring sequence at the 5′ or 3′ end of SEQ ID NO: 21, 22, or 23. Also, for example, a nucleic acid molecule of the invention can include one or more additional heterologous sequences such as, without limitation, nucleotide sequences encoding restriction enzyme sites or epitope tags. As non-limiting examples, nucleic acid molecules of the invention can be used in hybridization reactions such as Southern and Northern blots, to encode polypeptide sequence in recombinant cloning methods, or as primers in polymerase chain reactions.

The invention also provides an isolated nucleic acid molecule having a nucleotide sequence that encodes a polypeptide containing or consisting of substantially the same amino acid sequence as SEQ ID NO: 2, 4, 6, 8, 10, 12, 14 or 16. For example, the invention provides an isolated nucleic acid molecule having the nucleotide sequence of SEQ ID NO: 1, 3, 5, 7, 9, 11, 13 or 15.

The invention further provides a method for preventing or reducing the severity of a disease associated with an EP₄ receptor or EP₄ receptor variant in a subject by introducing into the subject a compound that modulates or specifically binds to an EP₄ receptor variant or another compound identified by a method of the invention. The invention also provides a method for regulating the circulatory system in a subject by introducing into the subject a compound that modulates or: specifically binds to an EP₄ receptor variant or another compound identified by a method of the invention. In addition, the invention provides a method for preventing or reducing the severity of cancer in a subject by introducing into the subject a compound that modulates or specifically binds to an EP₄ receptor variant or another compound identified by a method of the invention. Such a compound can be used, without limitation, to prevent or reduce the severity of colon cancer or cervical cancer. The invention further provides a method for preventing or reducing the severity of a bone disease such as osteoporosis in a subject by introducing into the subject a compound that modulates or specifically binds to an EP₄ receptor variant or another compound identified by a method of the invention.

As used herein, a “disease associated with an EP₄ receptor or EP₄ receptor variant” means any disease or condition in which modulation of the activity of the wild-type EP₄ receptor or EP₄ receptor variant can be beneficial. It is understood that the underlying cause of the disease may or may not be due to an abnormality in expression or activity of a wild-type EP₄ receptor or EP₄ receptor variant.

A disease associated with an EP₄ receptor or EP₄ receptor variant can be, without limitation, a circulatory system or cardiovascular disorder, an immune system disorder, cancer, osteoporosis, pain, or an ocular disorder such as glaucoma or ocular hypertension. As non-limiting examples, a compound identified according to a method of the invention can be used in humans for prophylatic treatment of a circulatory disorder or to prevent or reduce the severity of pain such as headache, muscle ache, or pain caused by any of a variety of inflammatory or degenerative joint diseases. In addition, a compound which modulate an EP₄ receptor variant can be used, without limitation, to modulate the female reproductive cycle or activity, for example, to induce labor, terminate pregnancy or regulate the female menstrual cycle.

A compound that modulates or differentially modulates an EP₄ receptor variant or another compound identified by a method of the invention can be useful for preventing or reducing the severity of a circulatory or cardiovascular disorder. Such circulatory or cardiovascular diseases include, but are not limited to, atherosclerosis; thrombosis; restenosis; vasculitis including autoimmune and viral vasculitis such as polyarteritis nodosa, Churg-Strass syndrome, ductus arteriousus, Takayasu's arteritis, Kawasaki Disease and Rickettsial vasculitis; atherosclerotic aneurisms; myocardial hypertrophy; congenital heart diseases (CHD); ischemic heart disease and anginas; acquired valvular/endocardial diseases; primary myocardial diseases including myocarditis; arrhythmias; and cardiac tumors. In addition, a compound that modulates or differentially modulates an EP₄ receptor variant or another compound identified by a method of the invention can be useful for preventing or reducing the severity of a disorder in the development of the circulatory system, for example, closure of the ductus arteriosus. Further, a compound that modulates or differentially modulates an EP₄ receptor variant or another compound identified by a method of the invention can be useful for preventing or reducing the severity of a disorder involving vasodilation of veins or arteries, for example, high blood pressure.

A compound that modulates or differentially modulates an EP₄ receptor variant or another compound identified by a method of the invention also can be useful for preventing or reducing the severity of an immune disease, which is a disease associated with inappropriate suppression or stimulation of the immune system. Immune diseases which can be prevented or reduced in severity according to a method of the invention include, without limitation, allergy and contact hypersensitivity. Diseases associated with stimulation of the immune system, including autoimmune diseases and other diseases associated with over-activation of the immune system, also can be prevented or reduced in severity according to a method of the invention.

A compound identified according to a method of the invention also can be used to prevent or reduce the severity of a cancer, which, as used herein, is a term that means any neoplastic disease including both solid tumors and hematopoietic cancers. Exemplary cancers to be prevented or reduced in severity according to a method of the invention include, without limitation, colon cancers; cervical cancers; melanomas, adenocarcinomas and other carcinomas; osteosarcomas; epithelial tumors such as breast and ovarian carcinomas; endometrial cancers; glioblastomas; renal cancers; bladder cancers; gastric cancers; pancreatic cancers; colorectal cancers; prostate cancers; lung cancers,.neuroblastomas; glioblastomas; leukemias and lymphomas; and vascular cell tumors such as hemangiomas, Kaposi's sarcomas, lymphangiomas, angiosarcomas and hemangioendotheliomas. It is understood that the methods of the invention can be used to prevent or reduce the severity of any of the above or other cancers known in the art, including cancers of various severities and stages. In one embodiment, a compound identified according to a method of the invention is used to prevent or reduce the severity of colon cancer. In a further embodiment, a compound identified according to a method of the invention is used to prevent or reduce the severity of cervical cancer.

A compound identified by the methods of the invention also can be used, without limitation, to prevent or reduce the severity of a bone disease. Such bone diseases include, but are not limited to, osteoporosis, Paget's disease, osteogenesis Imperfecta, bone infections, osteophytes (bone spurs), bone tumors, craniosynostosis and craniofacial disorders, osteopetrosis, primary hyperparathyroidism, Kippel-Feil syndrome, McCune-Albright syndrome myeloma bone disease and renal osteodystrophy.

The present invention also provides methods of preventing or reducing the severity of an ocular condition by administering a compound that modulates or differentially modulates an EP₄ receptor variant. Ocular conditions that can be prevented or reduced in severity with a compound that modulates or differentially modulates an EP₄ receptor variant include, without limitation, glaucoma, diabetic retinopathy; macular edema such as that associated with diabetes; conditions of retinal degeneration, for example, macular degeneration such as age-related macular degeneration (ARMD) and retinitis pigmentosa; retinal dystrophies; inflammatory disorders of the retina; vascular occlusive conditions of the retina such as retinal vein occlusions or branch or central retinal artery occlusions; retinopathy of prematurity; retinopathy associated with blood disorders such as sickle cell anemia; elevated intraocular pressure; ocular itch and discomfort; damage following retinal detachment; damage or insult due to vitrectomy, retinal or other surgery; and other retinal damage including therapeutic damage such as that resulting from laser treatment of the retina, for example, pan-retinal photocoagulation for diabetic retinopathy or photodynamic therapy of the retina, for example, for age-related macular degeneration. Ocular conditions that can be prevented or reduced in severity with a compound that modulates or differentially modulates an EP₄ receptor variant by a method of the invention further include, without limitation, genetic and acquired optic neuropathies such as optic neuropathies characterized primarily by loss of central vision, for example, Leber's hereditary optic neuropathy (LHON), autosomal dominant optic atrophy (Kjer disease) and other optic neuropathies such as those involving mitochondrial defects, aberrant dynamin-related proteins or inappropriate apoptosis; and optic neuritis such as that associated with multiple sclerosis, retinal vein occlusions or photodynamic or laser therapy. See, for example, Carelli et al., Neurochem. Intl. 40:573-584 (2002); and Olichon et al., J. Biol. Chem. 278:7743-7746 (2003).

In one embodiment, a compound identified by the methods of the invention is used to prevent or reduce the severity of glaucoma. Glaucoma, the second most common cause of blindness in the United States, affects about two million Americans, but roughly half are unaware of it. This group of disorders is characterized by progressive damage to the eye at least partly due to intraocular pressure. Normal intraocular pressure (IOP) ranges between 11 and 21 mm Hg; however, this level may not necessarily be healthy for all people. Some people with normal pressure develop optic nerve injury (normal- or low-pressure glaucoma). In contrast, many people have pressure greater than 21 mm Hg without any optic nerve injury (ocular hypertension). Of those with ocular hypertension, only about 1% per year will develop glaucoma.

Glaucoma can be described according to the mechanism of outflow obstruction as either open-angle or closed-angle (angle-closure) glaucoma. Alternatively, classification can be based on etiology as primary or secondary. The primary (conventional) outflow system of the eye is located in the anterior chamber angle and accounts for 83 to 96% of aqueous outflow in human eyes under normal circumstances. The primary outflow system refers to aqueous outflow through the trabecular meshwork, canal of Schlemm, intrascleral channels, and episcleral and conjunctival veins. In open-angle glaucoma with elevated intraocular pressure, pressure elevation occurs because outflow is inadequate despite an angle that appears open and relatively normal on gonioscopic examination. In closed-angle glaucoma, elevated intraocular pressure occurs when normal drainage of aqueous fluid from the eye is sufficiently prevented by a physical obstruction of the peripheral iris. The secondary (alternative) aqueous outflow pathways (known as the unconventional or uveoscleral aqueous outflow system) account for 5 to 15% of the total aqueous outflow. The secondary aqueous outflow pathway refers to aqueous exiting the eye through the anterior face of the ciliary body and percolating through the ciliary muscles to the suprachoroidal space (i.e., between the choroid and sclera), where it eventually exits the eye via scleral channels. It is understood that compounds that modulate or specifically bind to an EP₄ receptor variant or that are otherwise identified according to a method of the invention can be used to prevent or reduce the severity of any of a variety of forms of glaucoma including, but not limited to, normal- or low-pressure glaucoma, glaucoma with elevated intraocular pressure, primary glaucoma and secondary glaucoma.

Furthermore, a compound that modulates or specifically binds to an EP₄ receptor variant or which is otherwise identified by a method of the invention can be used alone or in combination with one or more different compounds or other therapeutics or procedures for treatment of glaucoma. Compounds that are currently used in the treatment of glaucoma include, but are not limited to, topical-blockers such as timolol, levobunolol, carteolol, metipranolol and betaxolol; topical nonselective adrenergic agonists such as epinephrine and dipivefrin; adrenergic agonists such as apraclonidine and brimonidine; topical cholinergic agonists such as pilocarpine and phospholine; oral carbonic anhydrase inhibitors such as acetazolamide and methazolamide; topical carbonic anhydrase inhibitors such as dorzolamide; and topical prostaglandin analogs such as latanoprost, unoprostone, and travoprost.

A compound that modulates or differentially modulates an EP₄ receptor variant or another compound identified by a method of the invention also can be useful for preventing or reducing the severity of pain. The term pain, as used herein, includes, without limitation, inflammatory pain, headache pain, muscle pain, visceral pain, neuropathic pain, and referred pain. Pain can be continuous or intermittent, of short duration such as acute pain, or of long duration such as chronic pain. Chronic pain is distinguished from acute pain, which is immediate, generally high threshold, pain brought about by injury such as a cut, crush, burn, or by chemical stimulation such as that experienced upon exposure to capsaicin, the active ingredient in chili peppers.

The methods of the invention further can be used, without limitation, to prevent or reduce the severity of chronic pain including headache pain such as pain associated with cluster headaches, tension headaches or chronic daily headaches; muscle pain including, but not limited to, that associated with back or other spasm; inflammatory pain or other symptoms resulting, for example, from spondylitis or arthritis such as rheumatoid arthritis, gouty arthritis, or osteoarthritis; gout; bursitis; painful menstruation and fever. In addition, the methods of the invention can be used, for example, to treat pain associated with injury, surgery, dental procedures, dysmenorrhea, labor and other pain associated with the female reproductive system, and systemic illness such as, without limitation, cancer. It is understood that these and other conditions which may respond to non-steroidal anti-inflammatory drugs (NSAIDs) can be prevented or reduced in severity using a compound that modulates or differentially modulates an EP₄ receptor variant.

In the methods of the invention for preventing or reducing the severity of any of the above disorders including, but not limited to, circulatory system and cardiovascular disorders, immune system disorders, cancers, bone diseases such as osteoporosis, ocular disorders and pain, the compound that modulates or differentially modulates an EP₄ receptor or receptor variant can optionally be formulated together with a pharmaceutically acceptable carrier for delivery to the subject to be treated. Suitable pharmaceutically acceptable carriers are well known in the art and include, for example, aqueous or organic solvents such as physiologically buffered saline, glycols, glycerol, oils or injectable organic esters. A pharmaceutically acceptable carrier can also contain a physiologically acceptable agent that acts, for example, to stabilize or increase solubility of a pharmaceutical composition. Such a physiologically acceptable agent can be, for example, a carbohydrate such as glucose, sucrose or dextrans; an antioxidant such as ascorbic acid or glutathione; a chelating agent; a low molecular weight polypeptide; or another stabilizer or excipient. Pharmaceutically acceptable carriers including solvents, stabilizers, solubilizers and preservatives, are well known in the art as described, for example, in Martin, Remington's Pharm. Sci. 15th Ed. (Mack Publ. Co., Easton, 1975).

Ophthalmic compositions can be useful in the methods of the invention for preventing or reducing the severity of an ocular condition. An ophthalmic composition contains an ophthalmically acceptable carrier, which is any carrier that has substantially no long term or permanent detrimental effect on the eye to which it is administered. Examples of ophthalmically acceptable carriers include, without limitation, water, such as distilled or deionized water; saline; and other aqueous media.

Topical ophthalmic compositions useful for preventing or reducing the severity of alleviating an ocular condition include, without limitation, ocular drops, ocular ointments, ocular gels and ocular creams. Such ophthalmic compositions are easy to apply and deliver the active compound effectively.

A preservative can be included, if desired, in an ophthalmic composition useful in a method of the invention. Such a preservative can be, without limitation, benzalkonium chloride, chlorobutanol, purite, thimerosal, phenylmercuric acetate, or phenylmercuric nitrate. Vehicles useful in a topical ophthalmic composition include, yet are not limited to, polyvinyl alcohol, povidone, hydroxypropyl methyl cellulose, poloxamers, carboxymethyl cellulose, hydroxyethyl cellulose and purified water.

A tonicity adjustor also can be included, if desired, in an ophthalmic composition. Such a tonicity adjustor can be, without limitation, a salt such as sodium chloride, potassium chloride, mannitol or glycerin, or another pharmaceutically or ophthalmically acceptable tonicity adjustor.

Various buffers and means for adjusting pH can be used to prepare an ophthalmic composition useful in the invention, provided that the resulting preparation is ophthalmically acceptable. Such buffers include, but are not limited to, acetate buffers, citrate buffers, phosphate buffers and borate buffers. It is understood that acids or bases can be used to adjust the pH of the composition as needed. Ophthalmically acceptable antioxidants useful in preparing an ophthalmic composition include, yet are not limited to, sodium metabisulfite, sodium thiosulfate, acetylcysteine, butylated hydroxyanisole and butylated hydroxytoluene.

Those skilled in the art can formulate a compound that modulates, differentially modulates, specifically binds, or differentially binds an EP₄ receptor variant to ensure proper compound distribution and bioavailablility in vivo. For example, some regions of the eye can be inaccessible to some systemically administered drugs, and as a result topical drug delivery can be used. Polymers can be added to ophthalmic solutions to increase bioavailability (Ludwig and Ootenhgm, S.T.P. Pharm. Sci. 2:81-87 (1992)). In addition, colloidal systems such as, without limitation, liposomes, microparticles or nanoparticules can be used to increase penetration of a compound into the eye. Ocular drug absorption also can be enhanced using, for example, iontophoresis, prodrugs, and cyclodextrins.

Methods of ensuring appropriate distribution in vivo also can be provided by rechargeable or biodegradable devices, particularly where concentration gradients or continuous delivery is desired. Various slow release polymeric devices are known in the art for the controlled delivery of drugs, and include both biodegradable and non-degradable polymers and hydrogels. Polymeric device inserts can allow for accurate dosing, reduced systemic absorption and in some cases, better patient compliance resulting from a reduced frequency of administration. Those skilled in the art understand that the choice of the pharmaceutical formulation and the appropriate preparation of the compound will depend on the intended use and mode of administration.

A compound that modulates or specifically binds to an EP₄ receptor variant, or that is otherwise identified by a screening method of the invention can be administered to a subject by any effective route. Suitable routes of administration include, but are not limited to, oral, topical, sublingual, intraocular, intradermal, parenteral, intranasal, intravenous, intramuscular, intraspinal, intracerebral and subcutaneous routes. The present invention also provides compounds containing an acceptable carrier such as any of the standard pharmaceutical carriers, including phosphate buffered saline solution, water and emulsions such as an oil and water emulsion, and various types of wetting agents.

A method of the invention can be practiced by peripherally administering to a subject an effective amount of a compound that modulates or differentially modulates an EP₄ receptor variant or another compound identified by a method of the invention,. As used herein in reference to such a compound, the term “peripherally administering” or “peripheral administration” means introducing the compound into a subject outside of the central nervous system. Thus, peripheral administration encompasses any route of administration other than direct administration to the spine or brain.

An effective amount of a compound of the invention can be administered to a subject by any of a variety of means depending, for example, on the type of condition to be treated, the pharmaceutical formulation, and the history, risk factors and symptoms of the subject. Routes of peripheral administration suitable for the methods of the invention include both systemic and local administration. As non-limiting examples, an effective amount of a compound of the invention can be administered orally; sublingually; parenterally; by subcutaneous pump; by dermal patch; by intravenous, intra-articular, subcutaneous or intramuscular injection; by topical drops, creams, gels or ointments; as an implanted or injected extended release formulation; or by subcutaneous minipump or other implanted device, and by inhalation by aerosol and similar devices.

One skilled in the art understands that peripheral administration can be local or systemic. Local administration results in significantly more of a compound of the invention being delivered to and about the site of local administration than to regions distal to the site of administration. Systemic administration results in delivery of a compound of the invention essentially throughout at least the entire peripheral system of the subject.

Routes of peripheral administration useful in the methods of the invention encompass, without limitation, oral administration, sublingual administration, topical administration, intravenous or other injection, and implanted minipumps or other extended release devices or formulations. A compound of the invention can be peripherally administered, without limitation, orally in any acceptable form such as in a tablet, pill, capsule, powder, liquid, suspension, emulsion or the like; as an aerosol; as a suppository; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation. A compound of the invention optionally can be packaged in unit dosage form suitable for single administration of precise dosages, or in sustained release dosage form for continuous controlled administration.

Chronic pain and other chronic conditions can be prevented or reduced in severity using any of a variety of forms of repeated or continuous administration as necessary. In the methods of the invention for preventing or reducing the severity of chronic pain or another chronic condition, means for repeated or continuous peripheral administration include, without limitation, repeated oral or topical administration, and administration via subcutaneous minipump.

It is understood that slow-release formulations can be useful in the methods of the invention, for example, for preventing or reducing the severity of chronic pain or another chronic condition. It is further understood that the frequency and duration of dosing will be dependent, in part, on the effect desired and the half-life of the modulating compound and that a variety of routes of administration are useful for delivering slow-release formulations, as detailed herein above.

An effective dose of a compound for use in a method of the invention can be determined, for example, by extrapolation from the concentration required in an EP₄ receptor or EP₄ receptor variant binding or activity assay such as one of the assays disclosed herein above. An effective dose of a compound for the treatment of a disease associated with an EP₄ receptor or EP₄ receptor variant also can be determined from appropriate animal models, such as transgenic animal models. As non-limiting examples, animal models for pathologies such as cardiovascular disease and ocular disease are well-known in the art. An effective dose for preventing or reducing the severity of a disease is a dose that results in either partial or complete alleviation of at least one symptom of the disease. The appropriate dose of a compound for treatment of a human subject can be determined by those skilled in the art, and is dependent, for example, on the particular disease being treated and its severity, the nature and bioactivity of the particular compound, the desired route of administration, the gender, age and general health of the individual, and the number of doses and duration of treatment.

All journal article, reference and patent citations provided herein, including referenced sequence accession numbers of nucleotide and amino acid sequences contained in various databases, in parentheses or otherwise, whether previously stated or not, are incorporated herein by reference in their entirety.

It is understood that modifications which do not substantially affect the activity of the various embodiments of this invention are also included within the definition of the invention provided herein. Accordingly, the following examples are intended to illustrate but not limit the present invention.

### EXAMPLE I

### Identification of Alternatively Spliced EP₄ Receptor Variants

This example describes the molecular cloning of several alternatively spliced EP₄ receptor variants and their expression in cell culture.

Total RNA derived from human heart, brain, lung, spleen, small intestine, skeletal muscle, kidney and liver tissue were purchased from Clontech. Total RNA was isolated from human eyes (NDRI; Philadelphia, Pa.) and human ocular tissues (ciliary smooth muscles, trabecular meshwork, ODM-2) using a Qiagen total RNA isolation kit, according to the manufacturer's instructions. The ODM-2 cell line is derived from human non-pigmented ciliary epithelial cells (Escribano et al., J. Cell. Physiol. 160:511-521 (1994)). Using 5 μg of human total RNA, first strand cDNA was synthesized using SuperScript II RNase H reverse transcriptase (Life Technologies; Carlsbad, Calif.). Reactions (20 μl) containing 5 μg of RNA, 250 ng of oligo (dT), and 100 units of reverse transcriptase were incubated at 42° C. for 1 hour and terminated by incubation at 100° C. for 3 minutes. The PCR buffer contained 10 mM Tris-HCl, pH 8.3, 50 mM KCl, 2 mM MgCl, 2.5 units AmpliTaq DNA polymerase, and 0.2 μM upstream and downstream primers in a final volume of 50 μl. After an initial incubation for 5 minutes at 94° C., samples were subjected to 30 cycles of 30 seconds at 95° C., 30 seconds at 58° C., and 30 seconds at 72° C. in a PE 9700 thermal cycler. The primers used for the detection of alternatively spliced EP₄ receptor variants were as follows:
Human EP₄ Forward: AGATGGTCATCTTACTCATTG (SEQ ID NO: 19) and
Human EP₄ Reverse: GATGTACTGCTGATCTCCTTC (SEQ ID NO: 20)

The PCR products were isolated from a 1.5% lower melting agarose gel, and subcloned into the TOPO PCRII vector (Invitrogen; Carlsbad, Calif.). Nucleotide sequencing of the vectors was performed by Sequetech (Mountain View, Calif.).

Full length cDNAs for EP₄ receptor variants VAR-1 to VAR-3 were isolated. The cDNAs are subcloned into TOPO pcDNA3.1 PCR cloning vector (Invitrogen; Carlsbad, Calif.) or pCEP4 expression vector (Invitrogen) to create Alt EP₄/pcDNA3.1 plasmids or Alt EP₄/pCEP4 plasmids. Alt EP₄/pcDNA3.1 plasmids are used for transient transfection, and Alt EP₄/pCEP4 plasmids are used for stable transfection. Full length Gα₁₆ cDNA is subcloned into the pcDNA3.1 vector.

HEK 293/EBNA cells are obtained from the American Type Culture Collection (ATCC) and routinely maintained in DMEM with 10% fetal bovine serum, 1% glutamine, 0.5% penicillin/streptomycin. Cells are kept in humidified 5% CO₂, 95% air at 37° C. For stable transfection, Alt EP₄/pCEP4 plasmids are transfected into HEK 293/EBNA cells using Fugene 6 (Roche Diagnostics Corp., Inc.; Indianapolis, Ind.), according to the manufacture's instructions, and then 200 mg/ml hygromycin is used to select cell clones that stably expressed the plasmid.

### EXAMPLE II

### Tissue Distribution of Alternatively Spliced EP₄ Receptor Variants

This example demonstrates the tissue distribution of alternatively spliced EP₄ receptor variants VAR-1 to VAR-3 using RT-PCR.

Human multiple tissue RNA samples were purchased from BD Biosciences (Clontech). Using 5 μg of human total RNA, first strand cDNA was synthesized by Superscript II RNase H reverse transcriptase (Life Technologies). Reactions (20 μl) containing 5 μl of RNA, 250 ng of oligo (dT), and 100 units of reverse transcriptase were incubated at 42° C. for 1 hour and terminated by 100° C. for 3 minutes.

PCR reactions contained the following: PCR buffer (10 mM Tris-HCl, pH 8.3, 50 mM KCl, 2 mM MgCl), 2.5 units AmpliTaq DNA polymerase, 0.2 μM forward and reverse primers, in a final volume of 50 μl. After an initial incubation for 5 minutes at 94° C., samples were subjected to 30 cycles of 30 seconds at 95° C., 30 seconds at 58° C., and 30 seconds at 72° C. in a PE 9700 thermal cycler. Multiple tissue RT-PCR analysis was performed to detect alternatively spliced EP₄ receptor variant mRNA using the following primers:

| | |
|---|---|
| VAR-1 and VAR-3 | |
| forward: TTACTCATTGCCAACCTCCCT | (SEQ ID NO: 24) |
| VAR-1 | |
| reverse: CCCAAATTTGTGGCATTTCT | (SEQ ID NO: 25) |
| VAR-3 | |
| reverse: CTACCCTGTAAGTTGCAGAATG | (SEQ ID NO: 26) |
| VAR-2 | |
| forward: TTTCCCAAGCTGCAAATGAA | (SEQ ID NO: 27) |
| reverse: TATCCAGGGGTCTAGGATGG | (SEQ ID NO: 28) |

As shown in FIG. 6, the EP₄ variants VAR-1, VAR-2 and VAR-3 were widely expressed in liver, kidney, brain, small intestine, spleen, lung, skeletal muscle, heart and eye.

### EXAMPLE III

### Screening Assays using Alternatively Spliced EP₄ Receptor Variants

This example describes FLIPR and luciferase assays for screening compounds against alternatively spliced EP₄ receptor variants.

HEK 293/EBNA cells transiently or stably expressing Alt EP₄/pcDNA3.1 plasmids are seeded at a density of 5×10³ cells per well in Biocoat® Poly-D-lysine-coated black-wall, clear-bottom 96-well plates (Becton-Dickinson; Franklin Lakes, N.J.) and allowed to attach overnight. At 48 hours after transfection, the cells are washed two times with HBSS-HEPES buffer (Hanks Balanced Salt Solution without bicarbonate and phenol red, 20 mM HEPES, pH 7.4) using a Lab Systems Cellwash plate washer. After 45 minutes of dye-loading in the dark, using the calcium-sensitive dye Fluo-4 AM at a final concentration of 2 mM, the plates are washed four times with HBSS-HEPES buffer to remove excess dye leaving 100 μl in each well. Plates are re-equilibrated to 37° C. for a few minutes. The cells are excited with an Argon laser at 488 nm, and emission is measured through a 510-570 nm bandwidth emission filter (FLIPR™; Molecular Devices; Sunnyvale, Calif.). Compound solution is added in a 50 μl volume to each well to give the desired final concentration. The peak increase in fluorescence intensity is recorded for each well. To generate concentration-response curves, compounds are tested in duplicate in a concentration range between 10⁻¹¹ and 10⁻⁵ M. The duplicate values are averaged.

CRE-luciferase reporter plasmids purchased from Invitrogen are used for detecting cAMP accumulation in Gₐₛ coupled receptors. pGL3-N-960 plasmids containing human Nur77 promoter (Uemura et al., J. Biol. Chem. 270:5427-5433 (1995)) and pGL3-CTGF-LUC plasmids containing human CTGF promoter are used for detecting calcium, PKC, and MAP kinase pathways associated with G_{aq} coupled receptors. For the pGL3-CTGF-LUC plasmid, a DNA fragment containing the CTGF promoter region from −2047 to +65 (Fu et al., J. Biol. Chem 276:45888-45894 (2001)) is cloned from human genomic DNA (Clontech). The fragment is subcloned into a pGL3 luciferase expression vector (Promega Inc.) creating the pGL3-CTGF-LUC plasmid.

Luciferase reporter plasmids are transfected into HEK 293/EBNA cells transiently or stably expressing alternatively spliced EP₄ receptor variants using Fugene 6, according to the manufacturer's instructions. In brief, the cells are plated in 24 well plates overnight, and then the 24 well plate cells are washed twice and resuspended in 1 ml of DMEM. The cell suspension is mixed with 0.2 μg of plasmid DNA in 100 μl of DMEM containing 0.6 μl Fugene 6 solution and added into each well. Plates are cultured for 24 hours at 37° C. before compounds are added to the cultures at concentrations ranging from 10⁻¹¹ to 10⁻⁶ M. Cells are harvested 6 hours later and lysed in 100 μl of 25 mM Tris-phosphate buffer (pH 7.5) containing 1% Triton X-100. Soluble extracts (20 μl) are assayed for luciferase activity as described below.

The luciferase assay is performed with a Promega assay kit (Promega, Inc.; Madison, Wis.) at room temperature using an Autolumat LB 953 (Berthold; Bad Wildbad, Germany). Luciferase content is measured by calculating the light emitted during the initial 10 seconds of the reaction. Relative luciferase activity is expressed as fold values of ratio compared to control. Experiments are independently repeated at least 3 times.

All journal article, reference and patent citations provided herein, including referenced sequence accession numbers of nucleotide and amino acid sequences contained in various databases, in parentheses or otherwise, whether previously stated or not, are incorporated herein by reference in their entirety.

Although the invention has been described with reference to the disclosed embodiments, those skilled in the art will readily appreciate that the specific experiments detailed are only illustrative of the invention. It should be understood that various modifications can be made without departing from the spirit of the invention.

1. An isolated polypeptide, comprising an amino acid sequence selected from SEQ ID NO: 10 or 16.

2. The isolated polypeptide of claim 1, comprising SEQ ID NO: 10.

3. The isolated polypeptide of claim 1, comprising SEQ ID NO: 16.

4. An isolated polypeptide, comprising a) an amino acid sequence having at least 50% amino acid identity with SEQ ID NO: 18, and b) an amino acid sequence selected from SEQ ID NOS: 8, 10, 12, 14, and 16, or a conservative variant thereof.

5. An isolated polypeptide, comprising an amino acid sequence selected from SEQ ID NOS: 2, 4 and 6, or a conservative variant thereof.

6. The isolated polypeptide of claim 5, wherein said polypeptide comprises an amino acid sequence selected from SEQ ID NOS: 2, 4 and 6.

7. The isolated polypeptide of claim 6, wherein said polypeptide consists of an amino acid sequence selected from SEQ ID NOS: 2, 4 and 6.

8. An EP₄ receptor variant binding agent, which binds SEQ ID NO: 10, or an epitope thereof.

9. The binding agent of claim 5, wherein said binding agent is an antibody, or antigen binding fragment thereof.

10. A cell, comprising the exogenously expressed polypeptide of claim 1, 4, or 5.

11. A method for identifying a compound that modulates an EP₄ receptor variant, comprising:
a) contacting said EP₄ receptor variant with a compound, wherein said EP₄ receptor variant is an isolated EP₄ receptor variant or an EP₄ receptor variant over-expressed in a genetically engineered cell, and
b) determining the level of an indicator, which correlates with modulation of said EP₄ receptor variant, wherein an alteration in the level of said indicator as compared to a control level indicates that said compound is a compound that modulates said EP₄ receptor variant.

12. The method of claim 11, wherein said alteration is an increase in the level of said indicator.

13. The method of claim 11, wherein said alteration is a decrease in the level of said indicator.

14. The method of claim 11, wherein said EP₄ receptor variant is a polypeptide comprising a) an amino acid sequence having at least 50% amino acid identity with SEQ ID NO: 18, and
b) an amino acid sequence selected from SEQ ID NOS: 8, 10, 12, 14, and 16, or a conservative variant thereof.

15. The method of claim 11, wherein said EP₄ receptor variant is a polypeptide comprising an amino acid sequence selected from SEQ ID NOS: 2, 4 and 6, or a conservative variant thereof.

16. The method of claim 11, wherein said EP₄ receptor variant is an isolated EP₄ receptor variant polypeptide.

17. The method of claim 11, wherein said EP₄ receptor variant is an EP₄ receptor variant over-expressed in a genetically engineered cell.

18. The method of claim 17, wherein said EP₄ receptor variant is exogenously expressed.

19. The method of claim 11, wherein said indicator is calcium.

20. The method of claim 11, wherein said compound is a polypeptide.

21. The method of claim 11, wherein said compound is a small molecule.

22. A method for identifying a compound that specifically binds to an EP₄ receptor variant, comprising:
a) contacting said EP₄ receptor variant with a compound, wherein said EP₄ receptor variant is an isolated EP₄ receptor variant or an EP₄ receptor variant over-expressed in a genetically engineered cell, and
b) determining specific binding of said compound to said EP₄ receptor variant.

23. The method of claim 22, wherein said EP₄ receptor variant is a polypeptide comprising
a) an amino acid sequence having at least 50% amino acid identity with SEQ ID NO: 18, and
b) an amino acid sequence selected from SEQ ID NOS: 8, 10, 12, 14, and 16, or a conservative variant thereof.

24. The method of claim 22, wherein said EP₄ receptor variant is a polypeptide comprising an amino acid sequence selected from SEQ ID NOS: 2, 4 and 6, or a conservative variant thereof.

25. The method of claim 22, wherein said EP₄ receptor variant is an isolated EP₄ receptor polypeptide.

26. The method of claim 22, wherein said EP₄ receptor is an EP₄ receptor variant over-expressed in a genetically engineered cell.

27. The method of claim 26, wherein said EP₄ receptor variant is exogenously expressed.

28. The method of claim 22, wherein said contacting occurs in vitro.

29. The method of claim 22, wherein said compound is a polypeptide.

30. The method of claim 22, wherein said compound is a small molecule.

31. A method for identifying a compound that differentially modulates an EP₄ receptor variant, comprising:
a) contacting said EP₄ receptor variant with a compound, wherein said EP₄ receptor variant is an isolated EP₄ receptor variant or an EP₄ receptor variant over-expressed in a genetically engineered cell;
b) determining the level of an indicator which correlates with modulation of said EP₄ receptor variant;
c) contacting a second receptor with said compound;
d) determining the level of a corresponding indicator which correlates with modulation of said second receptor; and
e) comparing the level of the indicator from step (b) with the level of the corresponding indicator from step (d), wherein a different level of the indicator from step (b) compared to the level of the corresponding indicator from step (d) indicates that said compound is a compound that differentially modulates said EP₄ receptor variant.

32. The method of claim 31, wherein said second receptor is a different EP₄ receptor variant.

33. The method of claim 31, wherein said second receptor comprises the amino acid sequence SEQ ID NO: 18, or a functional fragment thereof.

34. The method of claim 31, wherein the level of said indicator from step (b) is greater than the level of said corresponding indicator from step (d).

35. The method of claim 31, wherein the level of said indicator from step (b) is less than the level of said corresponding indicator from step (d).

36. The method of claim 31, wherein said EP₄ receptor variant is a polypeptide comprising
a) an amino acid sequence having at least 50% amino acid identity with SEQ ID NO: 18, and
b) an amino acid sequence selected from SEQ ID NOS: 8, 10, 12, 14, and 16, or a conservative variant thereof.

37. The method of claim 31, wherein said EP₄ receptor variant is a polypeptide comprising an amino acid sequence selected from SEQ ID NOS: 2, 4 and 6, or a conservative variant thereof.

38. The method of claim 31, wherein said EP₄ receptor variant is an isolated EP₄ receptor polypeptide.

39. The method of claim 31, wherein said EP₄ receptor variant is an EP₄ receptor variant over-expressed in a genetically engineered cell.

40. The method of claim 39, wherein said EP₄ receptor variant is exogenously expressed.

41. The method of claim 31, wherein said indicator in step (b) is calcium.

42. The method of claim 31, wherein said compound is a polypeptide.

43. The method of claim 31, wherein said compound is a small molecule.

44. A method for identifying a compound that differentially binds to an EP₄ receptor variant, comprising:
a) contacting said EP₄ receptor variant with a compound, wherein said EP₄ receptor variant is an isolated EP₄ receptor or an EP₄ receptor variant over-expressed in a genetically engineered cell;
b) determining specific binding of said compound to said EP₄ receptor variant;
c) contacting a second receptor with said compound;
d) determining specific binding of said compound to said second receptor; and
e) comparing the level of specific binding from step (b) with the level of specific binding from step (d), wherein a different level of specific binding from step (b) compared to the level of specific binding from step (d) indicates that said compound is a compound that differentially binds to said EP₄ receptor variant.

45. The method of claim 44, wherein said second receptor is a different EP₄ receptor variant.

46. The method of claim 44, wherein said second receptor comprises the amino acid sequence SEQ ID NO: 18, or a functional fragment thereof.

47. The method of claim 44, wherein said different level of specific binding is an increased level of binding.

48. The method of claim 44, wherein said different level of specific binding is a decreased level of binding.

49. The method of claim 44, wherein said EP₄ receptor variant is a polypeptide comprising
a) an amino acid sequence having at least 50% amino acid identity with SEQ ID NO: 18, and
b) an amino acid sequence selected from SEQ ID NOS: 8, 10, 12, 14, and 16, or a conservative variant thereof.

50. The method of claim 44, wherein said EP₄ receptor variant is a polypeptide comprising an amino acid sequence selected from SEQ ID NOS: 2, 4 and 6, or a conservative variant thereof.

51. The method of claim 44, wherein said EP₄ receptor variant is an isolated EP₄ receptor polypeptide.

52. The method of claim 44, wherein said EP₄ receptor variant is an EP₄ receptor variant over-expressed in a genetically engineered cell.

53. The method of claim 52, wherein said EP₄ receptor variant is exogenously expressed.

54. The method of claim 44, wherein said contacting occurs in vitro.

55. The method of claim 44, wherein said compound is a polypeptide.

56. The method of claim 44, wherein said compound is a small molecule.

57. An isolated nucleic acid molecule, comprising a nucleotide sequence that encodes a polypeptide comprising
a) an amino acid sequence having at least 50% amino acid identity with SEQ ID NO: 18, and
b) an amino acid sequence selected from SEQ ID NOS: 8, 10, 12, 14, and 16, or a conservative variant thereof.

58. An isolated nucleic acid molecule, comprising a nucleotide sequence that encodes an amino acid sequence selected from SEQ ID NOS: 2, 4 and 6, or a conservative variant thereof.

59. The isolated nucleic acid molecule of claim 55, wherein said isolated nucleic acid comprises a nucleotide sequence that encodes an amino acid sequence selected from SEQ ID NOS: 2, 4 and 6.

60. The isolated nucleic acid molecule of claim 59, wherein said isolated nucleic acid consists of a nucleotide sequence that encodes an amino acid sequence selected from SEQ ID NOS: 2, 4 and 6.

61. The isolated nucleic acid molecule of claim 58, wherein said nucleotide sequence is selected from SEQ ID NOS: 1, 3, and 5.

62. A vector, comprising the isolated nucleic acid molecule of claim 57 or 58.

63. A host cell, comprising the vector of claim 62.


SEQUENCE LISTING (28 sequences)

SEQ ID NO: 1 - DNA, 1559 bp, Homo sapiens
  CDS (1)...(1054)
atgtccactcccggggtcaattcgtccgcctccttgagccccgaccggctgaacagcccagtgaccatcccggcggtgatgttcatcttcggggtggtgggcaacctggtggccatcgtggtgctgtgcaagtcgcgcaaggagcagaaggagacgaccttctacacgctggtatgtgggctggctgtcaccgacctgttgggcactttgttggtgagcccggtgaccatcgccacgtacatgaagggccaatggcccgggggccagccgctgtgcgagtacagcaccttcattctgctcttcttcagcctgtccggcctcagcatcatctgcgccatgagtgtcgagcgctacctggccatcaaccatgcctatttctacagccactacgtggacaagcgattggcgggcctcacgctctttgcagtctatgcgtccaacgtgctcttttgcgcgctgcccaacatgggtctcggtagctcgcggctgcagtacccagacacctggtgcttcatcgactggaccaccaacgtgacggcgcacgccgcctactcctacatgtacgcgggcttcagctccttcctcattctcgccaccgtcctctgcaacgtgcttgtgtgcggcgcgctgctccgcatgcaccgccagttcatgcgccgcacctcgctgggcaccgagcagcaccacgcggccgcggccgcctcggttgcctcccggggccaccccgctgcctccccagccttgccgcgcctcagcgactttcggcgccgccggagcttccgccgcatcgcgggcgccgagatccagatggtcatcttactcattgccacctccctggtggtgctcatctgctccatcccgctcgtggcaaactggaagtggagtcataaaacaactgatgggatgtcagatgagtgtgaagcacatctatacatacaagagaaatgccacaaatttgggtgcgagtattcgtcaaccagttatatcagccaagtttggagcgagaagtcagtaaaaatccagatttgcaggccatccgaattgcttctgtgaaccccatcctagacccctggatatatatcctcctgagaaagacagtgctcagtaaagcaatagagaagatcaaatgcctcttctgccgcattggcgggtcccgcagggagcgctccggacagcactgctcagacagtcaaaggacatcttctgccatgtcaggccactctcgctccttcatctcccgggagctgaaggagatcagcagtacatctcagaccctcctgccagacctctcactgccagacctcagtgaaaatggccttggaggcaggaatttgcttccaggtgtgcctggcatgggcctggcccaggaagacaccacctcactgaggactttgcgaatatcagagacctcagactcttcacagggtcaggactcagagagtgtcttactggtggatgaggctggtgggagcggcagggctgggcctgcccctaaggggagctccctgcaagtcacatttcccagtgaaacactgaacttatcagaaaaatgtatataa

SEQ ID NO: 2 - protein, 350 aa, Homo sapiens
MSTPGVNSSASLSPDRLNSPVTIPAVMFIFGVVGNLVAIVVLCKSRKEQKETTFYTLVCGLAVTDLLGTLLVSPVTIATYMKGQWPGGQPLCEYSTFILLFFSLSGLSIICAMSVERYLAINHAYFYSHYVDKRLAGLTLFAVYASNVLFCALPNMGLGSSRLQYPDTWCFIDWTTNVTAHAAYSYMYAGFSSFLILATVLCNVLVCGALLRMHRQFMRRTSLGTEQHHAAAAASVASRGHPAASPALPRLSDFRRRRSFRRIAGAEIQMVILLIATSLVVLICSIPLVANWKWSHKTTDGMSDECEAHLYIQEKCHKFGCEYSSTSYISQVWSEKSVKIQICRPSELLL

SEQ ID NO: 3 - DNA, 1525 bp, Homo sapiens
  CDS (1)...(978)
atgtccactcccggggtcaattcgtccgcctccttgagccccgaccggctgaacagcccagtgaccatcccggcggtgatgttcatcttcggggtggtgggcaacctggtggccatcgtggtgctgtgcaagtcgcgcaaggagcagaaggagacgaccttctacacgctggtatgtgggctggctgtcaccgacctgttgggcactttgttggtgagcccggtgaccatcgccacgtacatgaagggccaatggcccgggggccagccgctgtgcgagtacagcaccttcattctgctcttcttcagcctgtccggcctcagcatcatctgcgccatgagtgtcgagcgctacctggccatcaaccatgcctatttctacagccactacgtggacaagcgattggcgggcctcacgctctttgcagtctatgcgtccaacgtgctcttttgcgcgctgcccaacatgggtctcggtagctcgcggctgcagtacccagacacctggtgcttcatcgactggaccaccaacgtgacggcgcacgccgcctactcctacatgtacgcgggcttcagctccttcctcattctcgccaccgtcctctgcaacgtgcttgtgtgcggcgcgctgctccgcatgcaccgccagttcatgcgccgcacctcgctgggcaccgagcagcaccacgcggccgcggccgcctcggttgcctcccggggccaccccgctgcctccccagccttgccgcgcctcagcgactttcggcgccgccggagcttccgccgcatcgcgggcgccgagatccagatggtcatcttactcattgccacctccctggtggtgctcatctgctccatcccgctcgtgtttcccaagctgcaaatgaagaatttatatcattctgcaacttacagggtagctataggtgcgagtattcgtcaaccagttatatcagccaagtttggagcgagaagtcagtgaaaatccagatttgcaggccatccgaattgcttctgtgaaccccatcctagacccctggatatatatcctcctgagaaagacagtgctcagtaaagcaatagagaagatcaaatgcctcttctgccgcattggcgggtcccgcagggagcgctccggacagcactgctcagacagtcaaaggacatcttctgccatgtcaggccactctcgctccttcatctcccgggagctgaaggagatcagcagtacatctcagaccctcctgccagacctctcactgccagacctcagtgaaaatggccttggaggcaggaatttgcttccaggtgtgcctggcatgggcctggcccaggaagacaccacctcactgaggactttgcgaatatcagagacctcagactcttcacagggtcaggactcagagagtgtcttactggtggatgaggctggtgggagcggcagggctgggcctgcccctaaggggagctccctgcaagtcacatttcccagtgaaacactgaacttatcagaaaaatgtatataa

SEQ ID NO: 4 - protein, 326 aa, Homo sapiens
MSTPGVNSSASLSPDRLNSPVTIPAVMFIFGVVGNLVAIVVLCKSRKEQKETTFYTLVCGLAVTDLLGTLLVSPVTIATYMKGQWPGGQPLCEYSTFILLFFSLSGLSIICAMSVERYLAINHAYFYSHYVDKRLAGLTLFAVYASNVLFCALPNMGLGSSRLQYPDTWCFIDWTTNVTAHAAYSYMYAGFSSFLILATVLCNVLVCGALLRMHRQFMRRTSLGTEQHHAAAAASVASRGHPAASPALPRLSDFRRRRSFRRIAGAEIQMVILLIATSLVVLICSIPLVFPKLQMKNLYHSATYRVAIGASIRQPVISAKFGARSQ

SEQ ID NO: 5 - DNA, 1768 bp, Homo sapiens
  CDS (1)...(981)
atgtccactcccggggtcaattcgtccgcctccttgagccccgaccggctgaacagcccagtgaccatcccggcggtgatgttcatcttcggggtggtgggcaacctggtggccatcgtggtgctgtgcaagtcgcgcaaggagcagaaggagacgaccttctacacgctggtatgtgggctggctgtcaccgacctgttgggcactttgttggtgagcccggtgaccatcgccacgtacatgaagggccaatggcccgggggccagccgctgtgcgagtacagcaccttcattctgctcttcttcagcctgtccggcctcagcatcatctgcgccatgagtgtcgagcgctacctggccatcaaccatgcctatttctacagccactacgtggacaagcgattggcgggcctcacgctctttgcagtctatgcgtccaacgtgctcttttgcgcgctgcccaacatgggtctcggtagctcgcggctgcagtacccagacacctggtgcttcatcgactggaccaccaacgtgacggcgcacgccgcctactcctacatgtacgcgggcttcagctccttcctcattctcgccaccgtcctctgcaacgtgcttgtgtgcggcgcgctgctccgcatgcaccgccagttcatgcgccgcacctcgctgggcaccgagcagcaccacgcggccgcggccgcctcggttgcctcccggggccaccccgctgcctccccagccttgccgcgcctcagcgactttcggcgccgccggagcttccgccgcatcgcgggcgccgagatccagatggtcatcttactcattgccacctccctggtggtgctcatctgctccatcccgctcgtggcaaactggaagtggagtcataaaacaactgatgggatgtcagatgagtgtgaagcacatctatacatacaagagaaatgccacaaatttgggtatgactatgacagaatctgaaagactggatttgttcaaataaatttggctaagtttaaaaaattgcaatgcctagaaggaagttttaaatcataacatgcaattgcagacccagtagtagtaacctgaagactattgagtaacataaaatttcccaagctgcaaatgaagaatttatatcattctgcaacttacagggtagctataggtgcgagtattcgtcaaccagttatatcagccaagtttggagcgagaagtcagtaaaaatccagatttgcaggccatccgaattgcttctgtgaaccccatcctagacccctggatatatatcctcctgagaaagacagtgctcagtaaagcaatagagaagatcaaatgcctcttctgccgcattggcgggtcccgcagggagcgctccggacagcactgctcagacagtcaaaggacatcttctgccatgtcaggccactctcgctccttcatctcccgggagctgaaggagatcagcagtacatctcagaccctcctgccagacctctcactgccagacctcagtgaaaatggccttggaggcaggaatttgcttccaggtgtgcctggcatgggcctggcccaggaagacaccacctcactgaggactttgcgaatatcagagacctcagactcttcacagggtcaggactcagagagtgtcttactggtggatgaggctggtgggagcggcagggctgggcctgcccctaaggggagctccctgcaagtcacatttcccagtgaaacactgaacttatcagaaaaatgtatataa

SEQ ID NO: 6 - protein, 326 aa, Homo sapiens [sequence omitted]

SEQ ID NO: 7 - DNA, 183 bp, Homo sapiens
  CDS (1)...(183)
gcaaactggaagtggagtcataaaacaactgatgggatgtcagatgagtgtgaagcacatctatacatacaagagaaatgccacaaatttgggtgcgagtattcgtcaaccagttatatcagccaagtttggagcgagaagtcagtaaaaatccagatttgcaggccatccgaattgcttctg

SEQ ID NO: 8 - protein, 61 aa, Homo sapiens
ANWKWSHKTTDGMSDECEAHLYIQEKCHKFGCEYSSTSYISQVWSEKSVKIQICRPSELLL

SEQ ID NO: 9 - DNA, 111 bp, Homo sapiens
  CDS (1)...(111)
tttcccaagctgcaaatgaagaatttatatcattctgcaacttacagggtagctataggtgcgagtattcgtcaaccagttatatcagccaagtttggagcgagaagtcag

SEQ ID NO: 10 - protein, 37 aa, Homo sapiens
FPKLQMKNLYHSATYRVAIGASIRQPVISAKFGARSQ

SEQ ID NO: 11 - DNA, 111 bp, Homo sapiens
  CDS (1)...(111)
gcaaactggaagtggagtcataaaacaactgatgggatgtcagatgagtgtgaagcacatctatacatacaagagaaatgccacaaatttgggtatgactatgacagaatc

SEQ ID NO: 12 - protein, 37 aa, Homo sapiens
ANWKWSHKTTDGMSDECEAHLYIQEKCHKFGYDYDRI

SEQ ID NO: 13 - DNA, 27 bp, Homo sapiens
  CDS (1)...(27)
atcccgctcgtggcaaactggaagtgg

SEQ ID NO: 14 - protein, 9 aa, Homo sapiens
IPLVANWKW

SEQ ID NO: 15 - DNA, 27 bp, Homo sapiens
  CDS (1)...(27)
atcccgctcgtgtttcccaagctgcaa

SEQ ID NO: 16 - protein, 9 aa, Homo sapiens
IPLVFPKLQ

SEQ ID NO: 17 - DNA, 1470 bp, Homo sapiens
  CDS (1)...(1470)
atgtccactcccggggtcaattcgtccgcctccttgagccccgaccggctgaacagcccagtgaccatcccggcggtgatgttcatcttcggggtggtgggcaacctggtggccatcgtggtgctgtgcaagtcgcgcaaggagcagaaggagacgaccttctacacgctggtatgtgggctggctgtcaccgacctgttgggcactttgttggtgagcccggtgaccatcgccacgtacatgaagggccaatggcccgggggccagccgctgtgcgagtacagcaccttcattctgctcttcttcagcctgtccggcctcagcatcatctgcgccatgagtgtcgagcgctacctggccatcaaccatgcctatttctacagccactacgtggacaagcgattggcgggcctcacgctctttgcagtctatgcgtccaacgtgctcttttgcgcgctgcccaacatgggtctcggtagctcgcggctgcagtacccagacacctggtgcttcatcgactggaccaccaacgtgacggcgcacgccgcctactcctacatgtacgcgggcttcagctccttcctcattctcgccaccgtcctctgcaacgtgcttgtgtgcggcgcgctgctccgcatgcaccgccagttcatgcgccgcacctcgctgggcaccgagcagcaccacgcggccgcggccgcctcggttgcctcccggggccaccccgctgcctccccagccttgccgcgcctcagcgactttcggcgccgccggagcttccgccgcatcgcgggcgccgagatccagatggtcatcttactcattgccacctccctggtggtgctcatctgctccatcccgctcgtggtgcgagtattcgtcaaccagttatatcagccaagtttggagcgagaagtcagtaaaaatccagatttgcaggccatccgaattgcttctgtgaaccccatcctagacccctggatatatatcctcctgagaaagacagtgctcagtaaagcaatagagaagatcaaatgcctcttctgccgcattggcgggtcccgcagggagcgctccggacagcactgctcagacagtcaaaggacatcttctgccatgtcaggccactctcgctccttcatctcccgggagctgaaggagatcagcagtacatctcagaccctcctgccagacctctcactgccagacctcagtgaaaatggccttggaggcaggaatttgcttccaggtgtgcctggcatgggcctggcccaggaagacaccacctcactgaggactttgcgaatatcagagacctcagactcttcacagggtcaggactcagagagtgtcttactggtggatgaggctggtgggagcggcagggctgggcctgcccctaaggggagctccctgcaagtcacatttcccagtgaaacactgaacttatcagaaaaatgtatataatag

SEQ ID NO: 18 - protein, 488 aa, Homo sapiens
MSTPGVNSSASLSPDRLNSPVTIPAVMFIFGVVGNLVAIVVLCKSRKEQKETTFYTLVCGLAVTDLLGTLLVSPVTIATYMKGQWPGGQPLCEYSTFILLFFSLSGLSIICAMSVERYLAINHAYFYSHYVDKRLAGLTLFAVYASNVLFCALPNMGLGSSRLQYPDTWCFIDWTTNVTAHAAYSYMYAGFSSFLILATVLCNVLVCGALLRMHRQFMRRTSLGTEQHHAAAAASVASRGHPAASPALPRLSDFRRRRSFRRIAGAEIQMVILLIATSLVVLICSIPLVVRVFVNQLYQPSLEREVSKNPDLQAIRIASVNPILDPWIYILLRKTVLSKAIEKIKCLFCRIGGSRRERSGQHCSDSQRTSSAMSGHSRSFISRELKEISSTSQTLLPDLSLPDLSENGLGGRNLLPGVPGMGLAQEDTTSLRTLRISETSDSSQGQDSESVLLVDEAGGSGRAGPAPKGSSLQVTFPSETLNLSEKCI

SEQ ID NO: 19 - DNA, 21 bp, Artificial Sequence
  primer
agatggtcatcttactcattg

SEQ ID NO: 20 - DNA, 21 bp, Artificial Sequence
  primer
gatgtactgctgatctccttc

SEQ ID NO: 21 - DNA, 20 bp, Homo sapiens
tcccgctcgtgtttcccaag

SEQ ID NO: 22 - DNA, 20 bp, Homo sapiens
gtagctataggtgcgagtat

SEQ ID NO: 23 - DNA, 20 bp, Homo sapiens
taacataaaatttcccaagc

SEQ ID NO: 24 - DNA, 21 bp, Artificial Sequence
  primer
ttactcattgccaacctccct

SEQ ID NO: 25 - DNA, 20 bp, Artificial Sequence
  primer
cccaaatttgtggcatttct

SEQ ID NO: 26 - DNA, 22 bp, Artificial Sequence
  primer
ctaccctgtaagttgcagaatg

SEQ ID NO: 27 - DNA, 20 bp, Artificial Sequence
  primer
tttcccaagctgcaaatgaa

SEQ ID NO: 28 - DNA, 20 bp, Artificial Sequence
  primer
tatccaggggtctaggatgg